(19) United States
(12) Patent Application Publication
(10) Pub. No.: US 2022/0133823 A1
(43) Pub. Date: May 5, 2022

(54) A MODIFIED ONCOLYTIC VIRUS, COMPOSITION AND USE THEREOF

(71) Applicant: GENESAIL BIOTECH (SHANGHAI) CO. LTD., Shanghai (CN)

(72) Inventor: Yi CEN, Shanghai (CN)

(21) Appl. No.: 17/274,435

(22) PCT Filed: Sep. 10, 2019
(86) PCT No.: PCT/CN2019/105139

§ 371 Date: Mar. 9, 2021

(30) Foreign Application Priority Data
Sep. 10, 2018 (CN) PCT/CN2018/104830

(51) Int. Cl. A61K 35/768; A61P 35/00; C07K 16/28; C12N 7/00

(52) U.S. Cl. CPC A61K 35/768; A61P 35/00; A61K 2039/507; C07K 16/2878; C12N 7/00; C07K 16/2818

(57) ABSTRACT

Provided is a modified oncolytic vims having a first heterologous polynucleotide encoding an immune checkpoint inhibitor and a second heterologous polynucleotide encoding an immune activator. Also provided is a pharmaceutical composition comprising the modified oncolytic virus and a method of treating a cancer comprising administering to a subject the modified oncolytic virus or the pharmaceutical composition.

### SEQUENCE LISTING

A copy of the Sequence Listing is submitted with the specification electronically via EFS-Web as an ASCII formatted sequence listing with a file name of “068615-8001US01-sequence list-20220109 ST25”, a creation date of Jan. 9, 2022, and a size of about 35 Kb. The sequence listing contained this ASCII formatted document is part of the specification and is herein incorporated by reference in its entirety.

### FIELD OF TECHNOLOGY

The present disclosure relates generally to modified oncolytic viruses, the composition comprising the modified oncolytic viruses and its use in the treatment of tumor.

### BACKGROUND OF THE INVENTION

Tumor is diagnosed in more than 14 million people every year worldwide. Despite of numerous advances in medical research, tumor accounts for approximately 16% of all deaths.

Malignant tumors are often resistant to conventional therapies and represent significant therapeutic challenges. For example, micro-metastasis can establish at a very early stage in the development of primary tumors. Therefore, at the time of diagnosis, many tumor patients already have microscopic metastasis. Tumor-reactive T cells can seek out and destroy these micro-metastasis and spare the surrounding healthy tissues. However, naturally existing T cell responses against malignancies are often not sufficient to cause regression of the primary or metastatic tumors.

Oncolytic viruses have shown potential as anti-tumor agents. Unlike conventional gene therapy, oncolytic viruses are able to spread through tumor tissue by virtue of viral replication and concomitant cell lysis. However, Oncolytic viruses itself are not sufficient to treat the primary or metastatic tumors either.

Therefore, the need for enhancing the potency of oncolytic viruses and clearing metastatic tumor cells is particularly acute.

### SUMMARY OF THE INVENTION

In one aspect, the present disclosure relates to a modified oncolytic virus comprising a virus genome having a first heterologous polynucleotide encoding an immune checkpoint inhibitor and a second heterologous polynucleotide encoding an immuno activator.

In certain embodiments, the oncolytic virus is selected from the group consisting of vaccinia, adenovirus, reovirus, measles, herpes simplex, Semliki Forest virus, Venezuelan equine encephalitis, Parvovirus, Chicken Anemia Virus, Measles Virus, Coxsackie Virus, Vesicular Stomatitis Virus, Seneca Valley Virus, Maraba virus and Newcastle disease virus. In certain embodiments, the oncolytic virus is derived from the Western Reserve strain.

In certain embodiments, the modified oncolytic virus is attenuated and can replicate in a tumor cell. In certain embodiments, the virus genome comprises at least one deletion or disruption that renders the virus capable of selective replication in a tumor cell. In certain embodiments, the deletion or the disruption is in an Open Reading Frame (ORF) encoding at least a part of an enzyme that is both essential for replication of the virus and preferentially expressed in a tumor cell than in a non-tumor cell. In certain embodiments, the enzyme is a kinase. In certain embodiments, the enzyme is thymidine kinase.

In certain embodiments, the immune checkpoint inhibitor is a first antibody capable of specifically binding to an immune checkpoint protein or the antigen binding fragment thereof. In certain embodiments, the immune checkpoint protein is selected from a group consisting of PD-1, PD-L1/2, CTLA-4, B7-H3/4, LAG3, TIM-3, VISTA and CD160.

In certain embodiments, the first antibody or the antigen binding fragment thereof specifically binds to SEQ ID NO: 1.

In certain embodiments, the first antibody or the antigen binding fragment thereof comprises a first heavy chain comprising SEQ ID NOs: 2, 3, and 4. In certain embodiments, the first heavy chain comprises a variable region having SEQ ID NO: 5 or a homologous sequence thereof having at least 80% sequence identity. In certain embodiments, the first heavy chain comprises an amino acid sequence of SEQ ID NO: 6 or a homologous sequence thereof having at least 80% sequence identity.

In certain embodiments, the first heterologous polynucleotide comprises a nucleic acid sequence of SEQ ID NO: 7 or a homologous sequence thereof having at least 80% sequence identity. In certain embodiments, the first heterologous polynucleotide comprises a nucleic acid sequence of SEQ ID NO: 8 or a homologous sequence thereof having at least 80% sequence identity.

In certain embodiments, the first antibody or the antigen binding fragment thereof further comprises a first light chain comprising SEQ ID NOs: 9, 10, and 11. In certain embodiments, the first light chain comprises a variable region having an amino acid sequence of SEQ ID NO: 12 or a homologous sequence thereof having at least 80% sequence identity. In certain embodiments, the first light chain comprises an amino acid sequence of SEQ ID NO: 13 or a homologous sequence thereof having at least 80% sequence identity.

In certain embodiments, the first heterologous polynucleotide further comprises a nucleic acid sequence of SEQ ID NO: 14 or a homologous sequence thereof having at least 80% sequence identity. In certain embodiments, the first heterologous polynucleotide further comprises a nucleic acid sequence of SEQ ID NO: 15 or a homologous sequence thereof having at least 80% sequence identity.

In certain embodiments, the immuno activator is a co-stimulatory activator. In certain embodiments, the immuno activator is a second antibody binding to a co-stimulatory molecule or the antigen binding fragment thereof.

In certain embodiments, the co-stimulatory molecule is selected from a group consisting of CD137 (4-1BB), CD27, CD70, CD86, CD80, CD28, CD40, CD122, TNFRS25, OX40, GITR, Neutrophilin and ICOS.

In certain embodiments, wherein the second antibody or the antigen binding fragment thereof specifically binds to SEQ ID NO: 16.

In certain embodiments, the second antibody or the antigen binding fragment thereof comprises a second heavy chain comprising SEQ ID NOs: 17, 18, and 19. In certain embodiments, the second heavy chain comprises a variable region having an amino acid sequence of SEQ ID NO: 20 or a homologous sequence thereof having at least 80% sequence identity. In certain embodiments, the second heavy chain comprises an amino acid sequence of SEQ ID NO: 21 or a homologous sequence thereof having at least 80% sequence identity.

In certain embodiments, the second heterologous polynucleotide comprises a nucleic acid sequence of SEQ ID NO: 22 or a homologous sequence thereof having at least 80% sequence identity. In certain embodiments, the second heterologous polynucleotide comprises a nucleic acid sequence of SEQ ID NO: 23 or a homologous sequence thereof having at least 80% sequence identity.

In certain embodiments, the second antibody or the antigen binding fragment thereof further comprises a second light chain comprising SEQ ID NOs: 24, 25, and 26. In certain embodiments, the second light chain comprises a variable region having an amino acid sequence of SEQ ID NO: 27 or a homologous sequence thereof having at least 80% sequence identity. In certain embodiments, the second heterologous polynucleotide further comprises a nucleic acid sequence of SEQ ID NO: 28 or a homologous sequence thereof having at least 80% sequence identity.

In certain embodiments, the second light chain comprises an amino acid sequence of SEQ ID NO: 29 or a homologous sequence thereof having at least 80% sequence identity.

In certain embodiments, the second heterologous polynucleotide further comprises a nucleic acid sequence of SEQ ID NO: 30 or a homologous sequence thereof having at least 80% sequence identity.

In certain embodiments, the immuno activator is a NK activator stimulating NK cell activity. In certain embodiments, the NK activator is a second antibody binding to NK molecule or the antigen binding fragment thereof. In certain embodiments, the NK molecule is selected from a group consisting of Siglec, TIGIT, KIRs and NKG2A/D.

In certain embodiments, the immuno activator is a macrophage activator stimulating macrophage cell activity. In certain embodiments, the macrophage activator is a second antibody binding to macrophage molecule or the antigen binding fragment thereof. In certain embodiments, the macrophage molecule is selected from a group consisting of CSF1R, CSF1 kinase, PS and CD47.

In certain embodiments, the immune checkpoint inhibitor is an antibody specifically binding to PD-1 or the antigen binding fragment thereof, and the immuno activator is an antibody specifically binding to CD137 or the antigen binding fragment thereof.

In certain embodiments, the first heterologous polynucleotide and the second heterologous polynucleotide is inserted in the place of the deletion.

In certain embodiments, there the first heterologous polynucleotide is immediately upstream or immediately downstream of the second heterologous polynucleotide.

In certain embodiments, the first heterologous polynucleotide encodes a first heavy chain and a first light chain of the first antibody. In certain embodiments, the first heterologous polynucleotide further comprises a first promoter capable of driving expression of the first heavy chain, and a second promoter capable of driving expression of the first light chain, wherein the first and the second promoters are in a head-to-head orientation.

In certain embodiments, the second heterologous polynucleotide encodes a second heavy chain and a second light chain of the second antibody. In certain embodiments, the second heterologous polynucleotide further comprises a third promoter capable of driving expression of the second heavy chain, and a fourth promoter capable of driving expression of the second light chain, wherein the third and the fourth promoters are in a head-to-head orientation.

In certain embodiments, the first heterologous polynucleotide and the second heterologous polynucleotide are configured such that they are expressed in the same or different stages of replicative cycle of the modified oncolytic virus.

In certain embodiments, the first and the second promoters are the same or different. In certain embodiments, the first and the second promoters are both later promoter. In certain embodiments, the later promoter is pSL.

In certain embodiments, the third and the fourth promoters are the same or different. In certain embodiments, the third and the fourth are both early and later promoter. In certain embodiments, the early and later promoter is pSE/L.

In certain embodiments, the modified oncolytic virus comprises the following elements in frame in an orientation from 5′ to 3′ of the sense strand: a polynucleotide encoding the light chain of an antibody binding to CD137-a first early and late promoter-a second early and late promoter-a polynucleotide encoding the heavy chain of an antibody binding to CD137-a polynucleotide encoding the heavy chain of an antibody binding to PD-1-a first late promoter-a second late promoter-a polynucleotide encoding the light chain of an antibody binding to PD-1.

In certain embodiments, the immune checkpoint inhibitor expressed from the first heterologous polynucleotide and the immuno activator expressed from the second heterologous polynucleotide are expressed as separate proteins.

In another aspect, the present disclosure relates to a pharmaceutical composition, comprising the modified oncolytic virus of the present disclosure and a pharmaceutically acceptable carrier.

In another aspect, the present disclosure relates to a method of treating a tumor, comprising administering to a subject an effective amount of the modified oncolytic virus of the present disclosure or the pharmaceutical composition of the present disclosure.

In certain embodiments, the subject is human.

In certain embodiments, the tumor is solid tumor. In certain embodiments, the tumor is melanoma, non-small cell lung cancer, renal cell carcinoma, Hodgkin lymphoma, squamous cell carcinoma of the head and neck, bladder cancer, colorectal cancer, or hepatocellular carcinoma.

In certain embodiments, the route of administering is topical. In certain embodiments, the route of administering is intra-tumor injection.

### BRIEF DESCRIPTION OF THE FIGURES

FIG. 1 shows the structure of thymidine kinase (TK) deletion, anti-PD-1 and anti-4-1BB antibodies insertion in WR-GS-600.

FIG. 2 shows the structure of TK deletion, and anti-PD-1 antibody insertion in WR-GS-620.

FIG. 3 is a schematic diagram of the recombination step for WR-GS-600.

FIG. 4 is a schematic diagram of the recombination step for WR-GS-620.

FIG. 5 shows primer location relative to GS-600 viral genome.

FIG. 6 shows primer location relative to GS-610 viral genome.

FIG. 7 shows primer location relative to GS-620 viral genome.

FIG. 8 shows alignment of WR-GS-600 with WR wild type strain.

FIG. 9 shows alignment of WR-GS-610 with WR wild type strain.

FIG. 10 shows alignment of WR-GS-620 with WR wild type strain.

FIG. 11 shows the result of immunofluorescence detection of human IgG expression. FIG. 11a shows the phase contrast image of U2OS cells. FIG. 11b shows background staining. FIG. 11c shows the image for WR-GS-600 infected U2OS cells. FIG. 11d shows the image for WR-GS-620 infected U2OS cells.

FIG. 12 shows Western blot result of human antibodies expressed by recombinant viruses (WR-GS-600, WR-GS-610 and WR-GS-620), using cell lysates, wherein P600, P610 and P620 refer to WR-GS-600, WR-GS-610 and WR-GS-620, respectively. This Western blotting experiment detects two bands with molecular weights of about 50 kDa and 25 kDa which correspond with the human antibody heavy chain and light chain, respectively.

FIG. 13 shows western blot result of human antibodies expressed by recombinant viruses (WR-GS-600, WR-GS-610 and WR-GS-620), using supernatants, wherein P600, P610 and P620 refer to WR-GS-600, WR-GS-610 and WR-GS-620, respectively. This Western blotting experiment detects two bands with molecular weights of about 50 kDa and 25 kDa which correspond with the human antibody heavy chain and light chain, respectively.

FIG. 14 shows bands resulted from PCR amplification using WR-GS-600, WR-GS-610 and WR-GS-620 viral DNA.

FIG. 15 shows amino acid sequences of heavy chain of anti-huPD-1 and its encoding sequence.

FIG. 16 shows amino acid sequences of light chain of anti-huPD-1 and its encoding sequence.

FIG. 17 shows amino acid sequences of heavy chain of anti-hu4-1BB and its encoding sequence.

FIG. 18 shows amino acid sequences of light chain of anti-hu4-1BB and its encoding sequence.

FIG. 19 shows ELISA result of PD-1 binding assay for WR-GS-620 infected supernatant.

FIG. 20 shows ELISA result of 4-1BB binding assay for WR-GS-600 infected supernatant and WR-GS-610 infected supernatant.

FIGS. 21-23 show viability of HCT-116, HT-29, MC-38 and CT-26 cells infected with WR, WR-GS-600, WR-GS-610 and WR-GS-620.

FIG. 24 shows plate setup for viral titer determination.

FIG. 25 shows a representative plate scan result of the WR-GS-610 viral titer determination.

FIG. 26 shows a representative plate scan result of the WR viral titer determination.

FIG. 27 shows a representative plate scan result of the WR-GS-620 viral titer determination.

FIG. 28 shows a representative plate scan result of the WR-GS-600 viral titer determination.

FIG. 29 shows a representative plate scan result for control group treated with formulation buffer (FB).

FIG. 30 shows in vivo viral distribution in tumor after intratumoral injection.

FIG. 31 shows in vivo viral distribution in ovary after intratumoral injection.

FIG. 32 shows in vivo viral distribution in brain, spleen, liver and lung after intratumoral injection. Integrated photon emission (1.928E10 versus 1.554E10) is considered proportional to the number of tumor cells. Based on the data, GS-600 controls tumor growth.

FIG. 33 shows tumor volume changes of syngeneic CT-26 murine tumor model after intratumoral injection (IT) of FB, WR, WR-GS-600, WR-GS-610 and WR-GS-620.

FIG. 34 shows efficacy model in syngeneic mouse model treated with different viruses.

FIGS. 35 and 36 show flow cytometry results of humanized HT-29-Luc subcutaneous tumor model intravenously injected with human PBMC.

FIGS. 37 and 38 show tumor volume changes of humanized HT-29 subcutaneous tumor model after intratumoral injection (IT) of FB, WR, WR-GS-600, WR-GS-610 and WR-GS-620.

FIG. 39 shows humanized HT-29-Luc subcutaneous tumor model with tumors stained, wherein the mice were not injected with hPBMC.

FIG. 40 shows humanized HT-29-Luc subcutaneous tumor model with tumors stained, wherein the mice were injected with hPBMC. Mice in Cage 2 were infected with WR-GS-600. Mice in Cage 3 were infected with WR. Mice in Cage 4 were infected with FB. Mice in Cage 5 were infected with WR-GS-610. In Cage 6, the first mouse was infected with WR-GS-600, the second mouse was infected with WR-GS-620, the third mouse was infected with WR-GS-610, the fourth mouse was infected with WR, and the fifth mouse was infected with FB. The mice in Cage 7 were infected with WR-GS-620.

FIG. 41 shows humanized HT-29-Luc intraperitoneal tumor model with tumors stained.

FIG. 42 shows percentage chemiluminescence intensity change after treatment with different viruses in a week.

### DETAILED DESCRIPTION

In one aspect, the present disclosure relates to a modified oncolytic virus comprising a virus genome inserted with a first heterologous polynucleotide encoding an immune checkpoint inhibitor and a second heterologous polynucleotide encoding an immuno activator.

Oncolytic Virus

The term “oncolytic virus” as used herein refers to a virus capable of selectively replicating in and slowing the growth or inducing the death of tumor cells, either in vitro or in vivo, while having no or minimal effect on normal cells. In certain embodiments, an oncolytic virus contains a viral genome packaged into a viral particle (or virion) and is infectious (i.e., capable of infecting and entering into a host cell or subject). In certain embodiments, the oncolytic virus can be a DNA virus or an RNA virus, and can be in any suitable form such as a DNA viral vector, a RNA viral vector or viral particles.

The term “selectively replicate” as used herein refers to that the replication rate of the oncolytic virus is significantly higher in tumor cells than in non-tumor cells (e.g. healthy cells). In certain embodiments, the oncolytic virus shows at least 50%, 60%, 70%, 80%, 90%, 1 fold, 2 folds, 3 folds, 4 folds, 5 folds, 10 folds, 50 folds, 100 folds or 1000 folds higher rate of lysis in tumor cells than in non-tumor cells (e.g., healthy cells).

In certain embodiments, the oncolytic virus of the present disclosure can selectively replicate in liver tumor cells (e.g., Hepal-6 cells, Hep3B cells, 7402 cells, and 7721 cells), breast tumor cells (e.g., MCF-7 cells), tongue tumor cells (e.g., TCa8113 cells), adenoid cystic tumor cells (e.g., ACC-M cells), prostate tumor cells (e.g., LNCaP cells), human embryo kidney cells (e.g., HEK293 cells), lung tumor cells (e.g., A549 cells), or cervical tumor cells (e.g., Hela cells).

The oncolytic viruses of the present disclosure can be derived from poxvirus (e.g., vaccinia virus), adenovirus (e.g., Delta-24, Delta-24-RGD, ICOVIR-5, ICOVIR-7, Onyx-015, ColoAdl, H101, and AD5/3-D24-GMCSF), reovirus (e.g., Reolysin), measles virus, herpes simplex virus (e.g., HSV, OncoVEX GMCSF), Newcastle Disease virus (e.g., 73-T PV701 and HDV-HUJ strains as well as those described in the following literatures: Phuangsab et al., 2001, Cancer Lett. 172(1): 27-36; Lorence et al., 2007, Curr. Cancer Drug Targets 7(2): 157-67; and Freeman et al., 2006, Mol. Ther. 13(1): 221-8), retrovirus (e.g., influenza virus), myxoma virus, rhabdovirus (e.g., vesicular stomatitis virus; those described in the following literatures: Stojdl et al., 2000, Nat. Med. 6(7): 821-5 and Stojdl et al., 2003, Cancer Cell 4(4): 263-75), picornavirus (e.g., Seneca Valley virus; SW-001 and NTX-010), coxsackievirus or parvovirus.

In certain embodiments, the oncolytic virus of the present disclosure is derived from a poxvirus. The term “poxvirus” as used herein refers to a virus belonging to the Poxviridae subfamily. In certain embodiments, the poxvirus is a virus belonging to the Chordopoxviridae subfamily. In certain embodiments, the poxvirus is a virus belonging to the Orthopoxvirus subfamily. Sequences of the genome of various poxviruses, for example, the vaccinia virus, cowpox virus, Canarypox virus, Ectromelia virus, Myxoma virus genomes are available in the art and specialized databases such as Genbank (accession number NC_006998, NC_003663, NC_005309, NC_004105, NC_001132, respectively).

In certain embodiments, the oncolytic virus of the present disclosure is derived from a vaccinia virus. Vaccinia viruses are members of the poxvirus family characterized by an approximately 190 kb double-stranded DNA genome that encodes numerous viral enzymes and factors that enable the virus to replicate independently from the host cell machinery. In certain embodiments, the vaccinia virus of the present disclosure is derived from Elstree, Copenhagen, Western Reserve or Wyeth strains. In certain embodiments, the vaccinia virus of the present disclosure is the Western Reserve strain. Western Reserve strain has been well characterized and its complete sequence is available on the NCBI site (www.ncbi.nlm.nih.gov) with access number of AY243312.

The term “modified oncolytic virus” as used herein refers to an oncolytic virus that has been modified by the introduction of a heterologous nucleic acid or protein or the alteration of a native nucleic acid or protein. In certain embodiments, the modified oncolytic virus provided herein is genetically altered by deletion and/or addition of nucleic acid sequences. In certain embodiments, the modified oncolytic virus provided herein comprises deletion of thymidine kinase (TK) gene. In certain embodiments, the modified oncolytic virus provided herein comprises addition of nucleic acid sequences encoding anti-human PD-1 and/or anti-human 4-1BB antibodies.

In certain embodiments, the modified oncolytic virus of the present disclosure is attenuated. In certain embodiments, the modified oncolytic virus has reduced (e.g. at least 90%, 80%, 70%, 60%, 50% less) or undetectable virulence compared to its wild type counterpart in the normal cells (e.g., healthy cells).

The modified oncolytic virus of the present disclosure can be derived from any oncolytic virus known in the art to be oncolytic by its propensity to selectivity replicate and kill tumor cells as compared to non-tumor cells. The oncolytic virus may be naturally oncolytic or may be rendered oncolytic by genetic engineering, such as by modifying one or more genes so as to increase tumor selectivity and/or preferential replication in tumor cells. Examples of such genes for modification include those involved in DNA replication, nucleic acid metabolism, host tropism, surface attachment, virulence, host cell lysis and virus spread (see for example Kirn et al., 2001, Nat. Med. 7: 781; Wong et al., 2010, Viruses 2: 78-106).

In certain embodiments, the virus genome of the modified oncolytic virus of the present disclosure comprises at least one deletion or disruption that renders the virus capable of selective replication in a tumor cell. For example, the deletion or disruption may reduce the expression or function of an enzyme essential for virus replication, such that the virus becomes less capable to replicate in the absence of such an enzyme. In some embodiments, the virus replication depends on the presence and/or level of such an enzyme in a cell, the higher the level of the enzyme, the higher replicate capability or rate of the virus.

In certain embodiments, the deletion or the disruption is in an Open Reading Frame (ORF). The term “open reading frame” or an “ORF” or “encoding sequence” as used herein refers to a DNA sequence that is capable of being translated into an amino acid sequence. An ORF usually begins with a start codon (e.g., ATG), followed by amino-acid encoding codons, and ends with a stop codon (e.g., TGA, TAA, TAG).

In certain embodiments, the ORF encodes at least a part of an enzyme that is essential for replication of the virus and is preferentially expressed in a tumor cell than in a non-tumor cell. The term “express” as used herein refers to a process wherein a protein or a peptide sequence is produced from its encoding DNA or RNA sequence. In certain embodiments, the enzyme is a kinase.

In certain embodiments, the deletion in the ORF constitutes 100%, more than 99%, more than 98%, more than 95%, more than 90%, more than 85%, more than 80%, more than 75%, more than 70%, more than 65%, more than 60%, more than 55%, more than 50%, more than 45%, more than 40%, more than 35%, more than 30%, more than 25%, more than 20%, more than 15%, or more than 10% of the full length of the ORF. In certain embodiments, the deletion in the ORF constitutes at least 10, 15, 20, 25, 30, 35, 40, 45, 50, 55, 60, 65, 70, 75, 80, 85, 90, 95, 100, 150, 200, 300, 500, 800, 1000, 1200, 1500, 1800, 2000, 2200, 2400, 2500 or more nucleotides (optionally contiguous).

In certain embodiments, the ORF for thymidine kinase (TK) is deleted or disrupted. TK is involved in the synthesis of deoxyribonucleotides. TK is needed for viral replication in normal cells as these cells have generally low concentration of nucleotides whereas it is dispensable in tumor cells, which contain high nucleotide concentration. In poxvirus, the thymidine kinase-encoding gene is located at locus J2R. In certain embodiments, TK is completely deleted.

In certain embodiments, the ORF of ribonucleotide reductase (RR) is deleted or disrupted. RR catalyzes the reduction of ribonucleotides to deoxyribonucleotides, which is a crucial step in DNA biosynthesis. The viral enzyme is composed of two heterologous subunits, designated R1 and R2, which are encoded respectively by the I4L and F4L locus. Sequences for the I4L and F4L genes and their locations in the genome of various poxvirus are available in public databases, for example via GenBank accession number DQ437594, DQ437593, DQ377804, AH015635, AY313847, AY313848, NC_003391, NC_003389, NC_003310, M-35027, AY243312, DQ011157, DQ011156, DQ011155, DQ011154, DQ011153, Y16780, X71982, AF438165, U60315, AF410153, AF380138, U86916, L22579, NC_006998, DQ121394 and NC_008291. In the context of the invention, either the I4L gene (encoding the R1 large subunit) or the F4L gene (encoding the R2 small subunit) or both may be deleted or disturbed.

In certain embodiments, the virus genome of the modified oncolytic virus further comprises an additional deletion or disruption that further increases the tumor-specificity of the virus. In certain embodiments, the additional deletion or disruption is in an ORF encoding at least part of a tumor-specific protein that is preferentially or specifically expressed in a tumor cell. A representative example of tumor-specific protein is VGF. VGF is a secreted protein which is expressed early after cell infection by virus and its function seems important for virus spread in normal cells. Another example is the A56R gene coding for hemagglutinin (Zhang et al., 2007, Cancer Res. 67: 10038-46). One further example is F2L gene which encodes the viral dUTPase involved in both maintaining the fidelity of DNA replication and providing the precursor for the production of TMP by thymidylate synthase (Broyles et al., 1993, Virol. 195: 863-5). Sequence of the vaccinia virus F2L gene is available in GenBank via accession number M25392.

Immune Checkpoint Inhibitor

The modified oncolytic virus provided herein comprises a virus genome having a first heterologous polynucleotide encoding for an immune checkpoint inhibitor.

The term “heterologous” as used herein means that the sequence is not endogenous to the wild type virus.

The term “encode” or “encoding for” as used herein refers to being capable of being transcribed into mRNA and/or translated into a peptide or protein.

The term “immune checkpoint protein” as used herein refers to a protein directly or indirectly involved in an immunological pathway that is important for preventing uncontrolled immune reactions and thus for the maintenance of self-tolerance and/or tissue protection. The one or more immune checkpoint modulator(s) as used herein may independently act at any step of the T cell-mediated immunity including clonal selection of antigen-specific cells, T cell activation, proliferation, trafficking to sites of antigen and inflammation, execution of direct effector function and signaling through cytokines and membrane ligands.

The term “immune checkpoint inhibitor” as used herein refers to a molecule capable of modulating the function of an immune checkpoint protein in a negative way. The immune checkpoint inhibitor can be of any one of the molecular modalities known in the art, including, but not limited to, aptamer, mRNA, siRNA, microRNA, shRNA, peptide, antibody, spherical nucleic acid, TALEN, Zinc Finger Nuclease, and CRISPR/Cas9.

In certain embodiments, the immune checkpoint inhibitor is a natural or engineered antagonist of an inhibitory immune checkpoint molecule, including, for example, ligands of CTLA-4 (e.g., B7.1, B7.2), ligands of TIM3 (e.g., Galectin-9), ligands of A2a Receptor (e.g., adenosine, Regadenoson), ligands of LAG3 (e.g., MHC class I or MHC class II molecules), ligands of BTLA (e.g., HVEM, B7-H4), ligands of KIR (e.g., MHC class I or MHC class II molecules), ligands of PD-1 (e.g., PD-L1, PD-L2), ligands of IDO (e.g., NKTR-218, Indoximod, NLG919).

In certain embodiments, the immune checkpoint inhibitor is an antibody (e.g. antagonist antibody) selected from the group consisting of anti-PD-1 (e.g., Nivolumab, Pidilizumab, Pembrolizumab, BMS-936559, BMS-936558, atezolizumab, Lambrolizumab, MK-3475, AMP-224, AMP-514, STI-A1110, TSR-042, or ANB011), anti-PD-L1 (e.g., KY-1003, MCLA-145, atezolizumab, MEDI-4736, MSB0010718C, STI-A1010, MPDL3280A, Dapirolizumab CDP-7657, MEDI-4920, or those recited in PCT/US2001/020964), anti-PD-L2, anti-(both PD-L1 and PD-L2) (e.g., AUR-012, and AMP-224), anti-CTLA-4 (e.g., Ipilimumab, Tremelimumab, or KAHR-102), anti-IDO (e.g., D-1-methyl-tryptophan (Lunate)), anti-KIR (e.g., Lirilumab, IPH2101, or IPH4102), anti-LAG3 (e.g., BMS-986016, IMP701, IMP321, or C9B7W), anti-TIM3 (e.g., F38-2E2 or ENUM005), anti-VISTA (e.g., VA.F6), anti-BTLA (e.g., AF3354), anti-CD73 (e.g., OSU-HDAC42 or MEDI-9447), anti-B7-H3 (e.g., MGA271, DS-5573a, or 8H9), anti-A2aR, anti-B7-1, anti-B7-H3 (e.g., MGA271), anti-B7-H4, anti-(both B7-H3 and B7-H4), anti-CD52 (e.g., alemtuzumab), anti-IL-10, anti-IL-35, anti-MICA (e.g., IPH43), and anti-CD39.

In certain embodiments, the immune checkpoint inhibitor is an antibody or the antigen binding fragment thereof capable of specifically binding to an immune checkpoint protein selected from a group consisting of PD-1, PD-L1/2, CTLA-4, B7-H3/4, LAG3, TIM-3, VISTA and CD160. In certain embodiments, the immune checkpoint inhibitor is an anti-PD-L1 or anti-PD-L2 antibody, or an inhibitor of both PD-L1 and PD-L2. In certain embodiments, the immune checkpoint inhibitor is an anti-B7-H3 or anti-B7-H4 antibody, or an inhibitor of both B7-H3 and B7-H4.

PD-1 Inhibitor

In certain embodiments, the first heterologous polynucleotide of the present disclosure encodes a PD-1 inhibitor.

The term “PD-1” as used herein refers to programmed cell death protein, which belongs to the superfamily of immunoglobulin and functions as coinhibitory receptor to negatively regulate the immune system. PD-1 is a member of the CD28/CTLA-4 family, and has two known ligands including PD-L1 and PD-L2. Representative amino acid sequence of human PD-1 is disclosed under the GenBank accession number: NP_005009.2, and the representative nucleic acid sequence encoding the human PD-1 is shown under the GenBank accession number: NM_005018.2.

PD-1 negatively modulates T cell activation, and this inhibitory function is linked to an immunoreceptor tyrosine-based inhibitory motif (ITIM) of its cytoplasmic domain (Parry et. al, 2005, Mol. Cell. Biol. 25:9543-53). Disruption of this inhibitory function of PD-1 can lead to autoimmunity. Sustained negative signals by PD-1 have been implicated in T cell dysfunctions in many pathologic situations, such as tumor immune evasion and chronic viral infections.

PD-1 inhibitor can be any agent inhibiting the activity of PD-1, such as those reduce the activity of PD-1 at least 5%, 10%, 20%, 40%, 50%, 80%, 90%, 95% or more.

The activity (e.g. of PD-1) may be reduced as a result of, for example, inhibition of binding between the functional protein and its ligand (e.g. binding between PD-1 and PD-L1), inhibition of its biological activation (e.g. PD-1's activation), and/or reduction of the level (e.g. PD-1 level).

In certain embodiments, the PD-1 inhibitor is an antibody (e.g. antagonistic antibody) capable of specifically binding to PD-1.

The term “specific binding” or “specifically binds” as used herein refers to a non-random binding reaction between two molecules, such as for example between an antibody and an antigen. In certain embodiments, the antibodies or antigen-binding fragments provided herein specifically bind human and/or monkey PD-1 with a binding affinity (KD) of ≤10⁻⁶ M (e.g., ≤5×10⁻⁷M, ≤2×10⁻⁷M, ≤10⁻⁷M, ≤5×10⁻⁸M, ≤2×10⁻⁸ M, ≤10⁻⁸M, ≤5×10⁻⁹ M, ≤2×10⁻⁹M, ≤10⁻⁹ M, ≤10⁻¹⁰ M). KD as used herein refers to the ratio of the dissociation rate to the association rate (k_{off}/kₒₙ), which may be determined using surface plasmon resonance methods for example using instrument such as Biacore.

In certain embodiments, the PD-1 inhibitor is a full length monoclonal antibody against PD-1.

In certain embodiments, the PD-1 antibody specifically binds to SEQ ID NO: 1.

In certain embodiments, the PD-1 antibody or the antigen binding fragment thereof comprises a first heavy chain comprising SEQ ID NOs: 2, 3, and 4.

The term “identity” as used herein, with respect to amino acid sequence (or nucleic acid sequence), refers to the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues are not considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

In certain embodiments, the heavy chain comprises a variable region having SEQ ID NO: 5 or a homologous sequence thereof having at least 80%, 85%, 90%, 92%, 93%, 94%, 95%, 96%, 97%, 98% or 99% sequence identity. In certain embodiments, the heavy chain comprises an amino acid sequence of SEQ ID NO: 6 or a homologous sequence thereof having at least 80% sequence identity.

In certain embodiments, the polynucleotide comprises a nucleic acid sequence of SEQ ID NO: 7 or a homologous sequence thereof having at least 80%, 85%, 90%, 92%, 93%, 94%, 95%, 96%, 97%, 98% or 99% sequence identity. In certain embodiments, the polynucleotide comprises a nucleic acid sequence of SEQ ID NO: 8 or a homologous sequence thereof having at least 80%, 85%, 90%, 92%, 93%, 94%, 95%, 96%, 97%, 98% or 99% sequence identity.

In certain embodiments, the PD-1 antibody or the antigen binding fragment thereof further comprises a light chain comprising SEQ ID NOs: 9,10, and 11. In certain embodiments, the light chain comprises a variable region having an amino acid sequence of SEQ ID NO: 12 or a homologous sequence thereof having at least 80%, 85%, 90%, 92%, 93%, 94%, 95%, 96%, 97%, 98% or 99% sequence identity. In certain embodiments, the light chain comprises an amino acid sequence of SEQ ID NO: 13 or a homologous sequence thereof having at least 80%, 85%, 90%, 92%, 93%, 94%, 95%, 96%, 97%, 98% or 99% sequence identity.

In certain embodiments, the polynucleotide further comprises a nucleic acid sequence of SEQ ID NO: 14 or a homologous sequence thereof having at least 80%, 85%, 90%, 92%, 93%, 94%, 95%, 96%, 97%, 98% or 99% sequence identity. In certain embodiments, the polynucleotide further comprises a nucleic acid sequence of SEQ ID NO: 15 or a homologous sequence thereof having at least 80%, 85%, 90%, 92%, 93%, 94%, 95%, 96%, 97%, 98% or 99% sequence identity.

Immuno Activator

The modified oncolytic virus provided herein comprises a virus genome having a second heterologous polynucleotide encoding for an immuno activator.

The term “immune activator” as used herein refers to any agent capable of enhancing immune system.

The term “enhance the immune system” as used herein refers to the ability of an agent to stimulate the generation of T cell activity, B cell activity, macrophage activity and/or NK cell activity.

In certain embodiments, the immuno activator is co-stimulatory activator, NK activator or macrophage activator.

Co-Stimulatory Molecule Activator

In certain embodiments, the immuno activator is a co-stimulatory molecule activator.

The term “co-stimulatory molecule” as used herein refers to cell surface molecules other than antigen receptors or Fc receptors that provide a second signal required for efficient activation and function of T lymphocytes upon binding to antigen. Examples of such co-stimulatory molecules include CD137 (i.e. 4-1BB), CD27, CD70, CD86, CD80, CD28, CD40, CD122, TNFRS25, OX40 (CD134), GITR, Neutrophilin, and ICOS (i.e. CD278).

In certain embodiments, the co-stimulatory activator can be a peptide, polypeptide (e.g. antibody) that can enhance the cellular immune system. In certain embodiments, the co-stimulatory activator is an antibody binding to a co-stimulatory molecule and thus stimulating the activity of the co-stimulatory molecule or the antigen binding fragment of such antibody, such as CD137 antibody (e.g., BMS-663513 or PF-05082566), CD28 antibody (e.g., TGN-1412), CD40 antibody (e.g., CP-870,893, CDX1140, BI-655064, BMS-986090, APX005, or APX005M), OX40 (CD 134) antibody (e.g., MEDI6383, MEDI6469, MEDI0562, or those described in U.S. Pat. No. 7,959,925), anti-GITR (e.g., TRX518, INBRX-110, or NOV-120301), CD70 antibody, CD86 antibody, CD80 antibody, CD122 antibody, TNFRS25 antibody, Neutrophilin antibody, and CD27 antibody (e.g., CDX-1127, BION-1402, or hCD27.15).

CD137 Activator

In certain embodiments, the second heterologous polynucleotide of the present disclosure encodes a CD137 activator.

CD137, also referred to as 4-1BB, is a member of the tumor necrosis factor receptor (TNFR) gene family which includes proteins involved in regulation of cell proliferation, differentiation, and programmed cell death (A. Ashkenazi, Nature, 2: 420-430, (2002)). CD137 is expressed predominantly on activated T cells, including both CD4⁺ and CD8⁺ cells, NK cells, and NK T cells (see B. Kwon et al., Mol. Cell 10: 119-126, (2000); J. Hurtado et al, J. Immunol. 155: 3360-3365, (1995); and L. Melero et al., Cell. Immunol. 190: 167-172, (1998)).

CD137 activator can be any agent enhancing the activity of PD-1, such as those enhance the activity of CD137 at least 5%, 10%, 20%, 40%, 50%, 80%, 90%, 95% or more.

In certain embodiments, the CD137 activator is an antibody specifically binding to CD137. In certain embodiments, the CD137 activator is a full length antibody.

In certain embodiments, the CD137 antibody or the antigen binding fragment thereof specifically binds to SEQ ID NO: 16.

In certain embodiments, the CD137 antibody or the antigen binding fragment thereof comprises a heavy chain comprising SEQ ID NOs: 17, 18, and 19.

In certain embodiments, the heavy chain comprises a variable region having an amino acid sequence of SEQ ID NO: 20 or a homologous sequence thereof having at least 80%, 85%, 90%, 92%, 93%, 94%, 95%, 96%, 97%, 98% or 99% sequence identity. In certain embodiments, the heavy chain comprises an amino acid sequence of SEQ ID NO: 21 or a homologous sequence thereof having at least 80%, 85%, 90%, 92%, 93%, 94%, 95%, 96%, 97%, 98% or 99% sequence identity.

In certain embodiments, the polynucleotide comprises a nucleic acid sequence of SEQ ID NO: 22 or a homologous sequence thereof having at least 80%, 85%, 90%, 92%, 93%, 94%, 95%, 96%, 97%, 98% or 99% sequence identity. In certain embodiments, the polynucleotide comprises a nucleic acid sequence of SEQ ID NO: 23 or a homologous sequence thereof having at least 80%, 85%, 90%, 92%, 93%, 94%, 95%, 96%, 97%, 98% or 99% sequence identity.

In certain embodiments, the antibody or the antigen binding fragment thereof further comprises a light chain comprising SEQ ID NOs: 24, 25, and 26. In certain embodiments, the light chain comprises a variable region having an amino acid sequence of SEQ ID NO: 27 or a homologous sequence thereof having at least 80%, 85%, 90%, 92%, 93%, 94%, 95%, 96%, 97%, 98% or 99% sequence identity. In certain embodiments, the polynucleotide further comprises a nucleic acid sequence of SEQ ID NO: 28 or a homologous sequence thereof having at least 80%, 85%, 90%, 92%, 93%, 94%, 95%, 96%, 97%, 98% or 99% sequence identity.

In certain embodiments, the light chain comprises an amino acid sequence of SEQ ID NO: 29 or a homologous sequence thereof having at least 80%, 85%, 90%, 92%, 93%, 94%, 95%, 96%, 97%, 98% or 99% sequence identity.

In certain embodiments, the polynucleotide further comprises a nucleic acid sequence of SEQ ID NO: 30 or a homologous sequence thereof having at least 80%, 85%, 90%, 92%, 93%, 94%, 95%, 96%, 97%, 98% or 99% sequence identity.

NK Activator

In certain embodiments, the immuno activator is a NK activator stimulating NK cell activity. In certain embodiments, the NK activator is a second antibody binding to NK molecule or the antigen binding fragment thereof.

In certain embodiments, the NK activator is selected from a group consisting of Siglec antibody, TIGIT antibody, KIRs antibody and NKG2A/D antibody (e.g., monalizumab).

Macrophage Activator

In certain embodiments, the immuno activator is a macrophage activator stimulating macrophage cell activity. In certain embodiments, the macrophage activator is a second antibody binding to macrophage molecule or the antigen binding fragment thereof.

In certain embodiments, the macrophage activator is selected from a group consisting of CSF1R antibody (e.g., FPA008), CSF1 kinase antibody, PS antibody and CD47 antibody (e.g., CC-90002, TTI-621, or VLST-007).

Antibody

The term “antibody” as used herein includes any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain, wherein the first constant region of the heavy chain is linked to the second constant region via a hinge region. The variable regions of the light and heavy chains are responsible for antigen binding specificity. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, and HCDR3). CDR boundaries for the antibodies and antigen-binding fragments disclosed herein may be defined or identified by the conventions of Kabat, Chothia, or Al-Lazikani (see Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991) for specifics). The three CDRs are interposed between flanking stretches known as framework regions (FRs), which are more highly conserved than the CDRs and form a scaffold to support the structure of the variable regions. The constant regions of the heavy and light chains are irrelevant to antigen binding specificity, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment formed from a portion of an antibody comprising one or more CDRs, but does not comprise an intact antibody structure. Examples of antigen-binding fragment include, without limitation, an Fab, an Fab′, an F(ab′)₂, an Fv fragment, a single-chain antibody molecule (scFv), an scFv dimer, a camelized single domain antibody, and a nanobody. An antigen-binding fragment is capable of binding to the same antigen to which the parent antibody binds.

The term “Fab” as used herein refers to that portion of the antibody consisting of a single light chain (both variable and constant regions) bound to the variable region and first constant region of a single heavy chain by a disulfide bond.

The term “Fab′” as used herein refers to a Fab fragment that includes a portion of the hinge region.

The term “F(ab′)₂” as used herein refers to a dimer of Fab′.

The term “Fv” as used herein refers to an Fv fragment consisting of the variable region of a single light chain and the variable region of a single heavy chain.

The term “Single-chain Fv antibody” or “scFv” as used herein refers to an engineered antibody consisting of a light chain variable region and a heavy chain variable region connected to one another directly or via a peptide linker sequence (see e.g., Huston J S et al., Proc Natl Acad Sci USA, 85:5879 (1988)).

The term “scFv dimer” as used herein refers to a polymer formed by two scFvs.

The term “camelized single domain antibody”, also known as “heavy chain antibody” or “HCAb” (heavy-chain-only antibody), refers to an antibody that contains two heavy chain variable regions but no light chains (see e.g., Riechmann L. and Muyldermans S., J Immunol Methods. December 10; 231(1-2):25-38 (1999); Muyldermans S., J Biotechnol. June; 74(4):277-302 (2001); WO94/04678; WO94/25591; and U.S. Pat. No. 6,005,079). Heavy chain antibodies were originally derived from Camelidae (camels, dromedaries, and llamas). Although devoid of light chains, camelized antibodies have an authentic antigen-binding repertoire (see Hamers-Casterman C. et al., Nature. 363(6428):446-8 (1993); Nguyen V K. et al., “Heavy-chain antibodies in Camelidae; a case of evolutionary innovation,” Immunogenetics. 54(1):39-47 (2002); and Nguyen V K. et al., Immunology. 109(1):93-101 (2003), which are incorporated herein by reference in their entirety).

The term “nanobody” as used herein refers to an antibody consisting of a heavy chain variable region from a heavy chain antibody and two constant regions, CH2 and CH3.

In certain embodiments, the antibody provided herein is a fully human antibody, a humanized antibody, a chimeric antibody, a mouse antibody or rabbit antibody. In certain embodiments, the antibody provided herein is a polyclonal antibody, a monoclonal antibody or a recombinant antibody. In certain embodiments, the antibody provided herein is a monospecific antibody, a bispecific antibody or a multispecific antibody. In certain embodiments, the antibody provided herein may further be labeled. In certain embodiments, the antibody or antigen-binding fragment thereof is a fully human antibody, which is optionally produced by a transgenic rat, e.g., a transgenic rat in which the expression of endogenous rat immunoglobin gene is inactivated, and carrying recombinant human immunoglobin locus with J loci deletions and C-kappa mutations, and which can also be expressed by an engineered cell (e.g., CHO cell).

The term “fully human” as used herein, with reference to antibody or antigen-binding fragment, refers to that the amino acid sequences of the antibody or the antigen-binding fragment correspond to that of an antibody produced by a human or a human immune cell, or derived from a non-human source such as a transgenic non-human animal that utilizes human antibody repertoires, or other human antibody-encoding sequences.

The term “humanized” as used herein, with reference to antibody or antigen-binding fragment, refers to an antibody or the antigen-binding fragment comprising CDRs derived from non-human animals, FR regions derived from human, and when applicable, constant regions derived from human. A humanized antibody or antigen-binding fragment is useful as human therapeutics in certain embodiments because it has reduced immunogenicity. In certain embodiments, the non-human animal is a mammal, for example, a mouse, a rat, a rabbit, a goat, a sheep, a guinea swine, or a hamster. In certain embodiments, the humanized antibody or antigen-binding fragment is composed of substantially all human sequences except for the CDR sequences which are non-human.

The term “chimeric” as used herein, with reference to antibody or antigen-binding fragment, refers to an antibody or antigen-binding fragment, having a portion of heavy and/or light chain derived from one species, and the rest of the heavy and/or light chain derived from a different species. In certain embodiments, a chimeric antibody may comprise a constant region derived from human and a variable region from a non-human species, such as from mouse or rabbit.

The term “conservative substitution” as used herein, with reference to amino acid sequence, refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

Polynucleotide

In certain embodiments, the modified oncolytic virus of the present disclosure contains a first heterologous polynucleotide that encodes an inhibitory antibody specifically binding to PD-1 or the antigen binding fragment thereof and a second heterologous polynucleotide that encodes an activating antibody specifically binding to CD137 or the antigen binding fragment thereof.

The term “polynucleotide” or “nucleic acid” as used herein refers to ribonucleic acids (RNA), deoxyribonucleic acids (DNA), or mixed ribonucleic acids-deoxyribonucleic acids such as DNA-RNA hybrids. The polynucleotide or nucleic acid may be single stranded or double stranded DNA or RNA or DNA-RNA hybrids. The polynucleotide or nucleic acid may be linear or circular. In certain embodiments, wherein the first and the second heterologous polynucleotide are both DNA when the virus is a DNA virus, or the first and the second heterologous polynucleotide are both RNA when the virus is a RNA virus. In certain embodiment, the first heterologous polynucleotide and the second heterologous polynucleotide are both double stranded DNA.

The first heterologous polynucleotide and the second heterologous polynucleotide may be introduced into the modified oncolytic virus using conventional methods known in the art, for example by synthesis by polymerase chain reaction (PCR) and ligation with the viral genome having compatible restriction ends. For more details, see, for example, Sambrook et al. Molecular Cloning: A Laboratory Manual (Cold Spring Harbor Laboratory, N.Y. (1989)), which is incorporated herein by reference in its entirety.

In certain embodiments, the first heterologous polynucleotide and the second heterologous polynucleotide is introduced in the place of the deletion in the ORF. In certain embodiments, the first heterologous polynucleotide is immediately upstream or immediately downstream of the second heterologous polynucleotide. The term “immediately upstream or immediately downstream” as used herein means the first heterologous polynucleotide and the second heterologous polynucleotide are located sufficiently close on the virus genome that they are separated from each other by no more than 0, 1, 2, 3, 4, 5, 6, 7, 8, 9, or 10 nucleotide(s). For example, the 3′ end of the upstream polynucleotide is immediately adjacent to the 5′ end of the downstream polynucleotide if the 3′ end of the upstream polynucleotide is separated from the 5′ end of the downstream polynucleotide by no more than 0, 1, 2, 3, 4, 5, 6, 7, 8, 9, or 10 nucleotide(s). In certain embodiments, there is no ORF between the first heterologous polynucleotide and the second heterologous polynucleotide. In certain embodiments, there is restriction site between the first heterologous polynucleotide and the second heterologous polynucleotide.

In certain embodiments, the first heterologous polynucleotide encodes a first heavy chain and a first light chain of the first antibody. In certain embodiments, the first heterologous polynucleotide further comprises a first promoter capable of driving expression of the first heavy chain, and a second promoter capable of driving expression of the first light chain, wherein the first and the second promoters are in a head-to-head orientation.

In certain embodiments, the first heterologous polynucleotide encodes a variable region of first heavy chain of the first antibody, a linker and a variable region of first light chain of the first antibody. In certain embodiments, the first heterologous polynucleotide encodes the first heavy chain of the first antibody, but does not encode the first light chain of the first antibody.

The term “head-to-head orientation” as used herein means that two promoters are immediately adjacent to each other on the virus genome and they drive protein expression in opposite directions. An illustrative example is shown in FIG. 2.

In certain embodiments, the second heterologous polynucleotide encodes a second heavy chain and a second light chain of the second antibody. In certain embodiments, the second heterologous polynucleotide further comprises a third promoter capable of driving expression of the second heavy chain, and a fourth promoter capable of driving expression of the second light chain, wherein the third and the fourth promoters are in a head-to-head orientation.

The term “promoter” as used herein refers to a polynucleotide sequence that can control transcription of an encoding sequence. The promoter sequence includes specific sequences that are sufficient for RNA polymerase recognition, binding and transcription initiation. In addition, the promoter sequence may include sequences that modulate this recognition, binding and transcription initiation activity of RNA polymerases. The promoter may affect the transcription of a gene located on the same nucleic acid molecule as itself or a gene located on a different nucleic acid molecule as itself. Functions of the promoter sequences, depending upon the nature of the regulation, may be constitutive or inducible by a stimulus. A “constitutive” promoter as used herein refers to a promoter that functions to continually activate gene expression in host cells. An “inducible” promoter as used herein refers to a promoter that activates gene expression in host cells in the presence of certain stimulus or stimuli.

In certain embodiments, the promoters of the present disclosure are eukaryotic promoters such as the promoters from CMV (e.g., the CMV immediate early promoter (CMV promoter)), epstein barr virus (EBV) promoter, human immunodeficiency virus (HIV) promoter (e.g., the HIV long terminal repeat (LTR) promoter), moloney virus promoter, mouse mammary tumor virus (MMTV) promoter, rous sarcoma virus (RSV) promoter, SV40 early promoter, promoters from human genes such as human myosin promoter, human hemoglobin promoter, human muscle creatine promoter, human metalothionein beta-actin promoter, human ubiquitin C promoter (UBC), mouse phosphoglycerate kinase 1 promoter (PGK), human thymidine kinase promoter (TK), human elongation factor 1 alpha promoter (EF1A), cauliflower mosaic virus (CaMV) 35S promoter, E2F-1 promoter (promoter of E2F1 transcription factor 1), promoter of alpha-fetoprotein, promoter of cholecystokinin, promoter of carcinoembryonic antigen, promoter of C-erbB2/neu oncogene, promoter of cyclooxygenase, promoter of CXC-Chemokine receptor 4 (CXCR4), promoter of human epididymis protein 4 (HE4), promoter of hexokinase type II, promoter of L-plastin, promoter of mucin-like glycoprotein (MUC1), promoter of prostate specific antigen (PSA), promoter of survivin, promoter of tyrosinase related protein (TRP1), and promoter of tyrosinase.

In certain embodiments, the promoters of the present disclosure may be tumor specific promoters. The term “tumor specific promoter” as used herein refers to a promoter that functions to activate gene expression preferentially or exclusively in tumor cells, and has no activity or reduced activity in non-tumor cells or non-tumor cells. Illustrative examples of tumor specific promoters include, without limitation, E2F-1 promoter, promoter of alpha-fetoprotein, promoter of cholecystokinin, promoter of carcinoembryonic antigen, promoter of C-erbB2/neu oncogene, promoter of cyclooxygenase, promoter of CXCR4, promoter of HE4, promoter of hexokinase type II, promoter of L-plastin, promoter of MUC1, promoter of PSA, promoter of survivin, promoter of TRP1, and promoter of tyrosinase.

In certain embodiments, the first heterologous polynucleotide and the second heterologous polynucleotide are configured such that they are expressed in the same or different stages of replicative cycle of the modified oncolytic virus. For example, the two polynucleotides may be both driven by early promoters which are induced at an early stage of virus replication, or alternatively both driven by later promoters which are induced at a late stage of virus replication, or alternatively one is driven by an early promoter, and the other is driven by a later promoter.

In certain embodiments, the first and the second promoters are the same or different. In certain embodiments, the first and the second promoters are both later promoter. In certain embodiments, the later promoter is pSL.

In certain embodiments, the third and the fourth promoters are the same or different. In certain embodiments, the third and the fourth are both early and later promoter. In certain embodiments, the early and later promoter is pSE/L.

In certain embodiments, the modified oncolytic virus comprises the following elements in frame in an orientation from 5′ to 3′ of the sense strand: a polynucleotide encoding the light chain of an antibody binding to CD137-a first early and late promoter-a second early and late promoter-a polynucleotide encoding the heavy chain of the antibody binding to CD137-a polynucleotide encoding the heavy chain of antibody binding to PD-1-a first late promoter-a second late promoter-a polynucleotide encoding the light chain of an antibody binding to PD-1.

In certain embodiments, the immune checkpoint inhibitor expressed from the first heterologous polynucleotide and the immuno activator expressed from the second heterologous polynucleotide are expressed as separate proteins. In other words, they are not expressed as a fusion protein, and are not connected with each other either (whether covalently or through a linker). In certain embodiments, the immune checkpoint inhibitor expressed from the first heterologous polynucleotide is not fused with any other protein and the immuno activator expressed from the second heterologous polynucleotide is not fused with any other protein.

In certain embodiments, the modified oncolytic virus does not include any other heterologous polynucleotides that encode immune checkpoint inhibitor or immuno activator, except for the first heterologous polynucleotide and the second heterologous polynucleotide. In certain embodiments, the modified oncolytic virus does not include any other protein encoding heterologous polynucleotides except for the first heterologous polynucleotide and the second heterologous polynucleotide.

Pharmaceutical Composition

In another aspect, the present disclosure provides a pharmaceutical composition, comprising the modified oncolytic virus described in the present disclosure and a pharmaceutically acceptable carrier.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In certain embodiments, compounds, materials, compositions, and/or dosage forms that are pharmaceutically acceptable refer to those approved by a regulatory agency (such as U.S. Food and Drug Administration, China Food and Drug Administration or European Medicines Agency) or listed in generally recognized pharmacopoeia (such as U.S. Pharmacopoeia, China Pharmacopoeia or European Pharmacopoeia) for use in animals, and more particularly in humans.

The pharmaceutically acceptable carriers for use in the pharmaceutical compositions of the present invention may include, but are not limited to, for example, pharmaceutically acceptable liquids, gels, or solid carriers, aqueous vehicles (e.g., sodium chloride injection, Ringer's injection, isotonic glucose injection, sterile water injection, or Ringer's injection of glucose and lactate), non-aqueous vehicles (e.g., fixed oils of vegetable origin, cottonseed oil, corn oil, sesame oil, or peanut oil), antimicrobial agents, isotonic agents (such as sodium chloride or dextrose), buffers (such as phosphate or citrate buffers), antioxidants (such as sodium bisulfate), anesthetics (such as procaine hydrochloride), suspending/dispending agents (such as sodium carboxymethylcellulose, hydroxypropyl methylcellulose, or polyvinylpyrrolidone), chelating agents (such as EDTA (ethylenediamine tetraacetic acid) or EGTA (ethylene glycol tetraacetic acid)), emulsifying agents (such as Polysorbate 80 (Tween-80)), diluents, adjuvants, excipients, or non-toxic auxiliary substances, other components known in the art, or various combinations thereof. Suitable components may include, for example, fillers, binders, disintegrants, buffers, preservatives, lubricants, flavorings, thickeners, coloring agents, or emulsifiers.

In certain embodiments, the pharmaceutical composition is an oral formulation. The oral formulations include, but are not limited to, capsules, cachets, pills, tablets, troches (for taste substrates, usually sucrose and acacia or tragacanth), powders, granules, or aqueous or non-aqueous solutions or suspensions, or water-in-oil or oil-in-water emulsions, or elixirs or syrups, or confectionery lozenges (for inert bases, such as gelatin and glycerin, or sucrose or acacia) and/or mouthwash and its analogs.

In certain embodiments, the pharmaceutical composition may be an injectable formulation, including sterile aqueous solutions or dispersions, suspensions or emulsions. In all cases, the injectable formulation should be sterile and should be liquid to facilitate injections. It should be stable under the conditions of manufacture and storage, and should be resistant to the infection of microorganisms (such as bacteria and fungi). The carrier may be a solvent or dispersion medium containing, for example, water, ethanol, polyols (e.g., glycerol, propylene glycol, and liquid polyethylene glycols, etc.) and suitable mixtures and/or vegetable oils thereof. The injectable formulation should maintain proper fluidity, which may be maintained in a variety of ways, for example, using a coating such as lecithin, using a surfactant, etc. Antimicrobial contamination can be achieved by the addition of various antibacterial and antifungal agents (e.g., parabens, chlorobutanol, phenol, sorbic acid, thimerosal, etc.).

In certain embodiments, unit-dose parenteral preparations are packaged in an ampoule, a vial or a syringe with a needle. All preparations for parenteral administration should be sterile and not pyretic, as is known and practiced in the art.

Method of Treatment

In another aspect, the present disclosure provides a method of treating a tumor, comprising administering to a subject an effective amount of the modified oncolytic virus of the present disclosure or the pharmaceutical composition of the present disclosure.

The term “subject” as used herein refers to human and non-human animal. Non-human animals include all vertebrates, e.g., mammals and non-mammals. A “subject” may also be a livestock animal (e.g., cow, swine, goat, chicken, rabbit or horse), or a rodent (e.g., rat or mouse), or a primate (e.g., gorilla or monkey), or a domestic animal (e.g., dog or cat). A “subject” may be a male or a female, and also may be at different ages. In certain embodiments, the subject is a human. A human “subject” may be Caucasian, African, Asian, Sumerian, or other races, or a hybrid of different races. A human “subject” may be elderly, adult, teenager, child or infant.

The term “tumor” as used herein refers to any medical condition mediated by neoplastic or malignant cell growth, proliferation, or metastasis, and includes both solid tumors and non-solid tumors such as leukemia. In the present disclosure, “tumor” is used interchangeably with the terms “cancer”, “malignancy”, “hyperproliferation” and “neoplasm(s)”. The term “tumor cell(s)” is interchangeable with the terms “cancer cell(s)”, “malignant cell(s)”, “hyperproliferative cell(s)”, and “neoplastic cell(s)” unless otherwise explicitly indicated. In certain embodiments, the tumor is selected from the group consisting of head and neck tumor, breast tumor, colorectal tumor, liver tumor, pancreatic adenocarcinoma, gallbladder and bile duct tumor, ovarian tumor, cervical tumor, small cell lung tumor, non-small cell lung tumor, renal cell carcinoma, bladder tumor, prostate tumor, bone tumor, mesothelioma, brain tumor, soft tissue sarcoma, uterine tumor, thyroid tumor, nasopharyngeal carcinoma, and melanoma. In certain embodiments, the tumor is solid tumor. In certain embodiments, the tumor is melanoma, non-small cell lung cancer, renal cell carcinoma, Hodgkin lymphoma, squamous cell carcinoma of the head and neck, bladder cancer, colorectal cancer, or hepatocellular carcinoma. In certain embodiments, the tumor has been refractory to prior therapy (e.g., administration of oncolytic virus, immune checkpoint inhibitor and/or immuno activator separately).

The term “treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to tumor, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

The modified oncolytic virus and the pharmaceutical composition may be administered via any suitable routes known in the art, including without limitation, parenteral, oral, enteral, buccal, nasal, topical, rectal, vaginal, transmucosal, epidermal, transdermal, dermal, ophthalmic, pulmonary, and subcutaneous administration routes. In certain embodiments, the route of administering is topical. In certain embodiments, the route of administering is intra-tumor injection.

In certain embodiments, the modified oncolytic virus and the pharmaceutical composition is administered at a therapeutically effective dosage. The term “therapeutic effective dosage” as used herein refers to the amount of a drug capable of ameliorating or eliminating a disease or symptom of a subject, or of preventively inhibiting or preventing the occurrence of the disease or symptom. A therapeutically effective amount can be the amount of a drug that ameliorates one or more diseases or symptoms of a subject to certain extent; the amount of a drug capable of restoring one or more physiological or biochemical parameters associated with the cause of a disease or symptom, partly or completely back to normal; and/or the amount of a drug capable of reducing the possibility that a disease or symptom occurs.

The therapeutically effective dosage of the modified oncolytic virus and the pharmaceutical composition is dependent on various factors known in the art, for example, body weight, age, pre-existing medical condition, therapy currently being received, health condition of the subject, and intensity, allergic, superallergic and side effect of drug interaction, and route of administration and the extent to which the disease develops. A skilled artisan (e.g., a physician or veterinarian) may reduce or increase dosage in accordance with these or other conditions or requirement.

In certain embodiments, the modified oncolytic virus and the pharmaceutical composition may be administered at a therapeutically effective dosage of about 10⁴ PFU to about 10¹⁴ PFU (e.g., about 10⁴ PFU, about 2*10⁴ PFU, about 5*10⁴ PFU, about 10⁵ PFU, about 2*10⁵ PFU, about 5*10⁵ PFU, about 10⁶ PFU, about 2*10⁶ PFU, about 5*10⁶ PFU, about 10⁷ PFU, about 2*10⁷ PFU, about 5*10⁷ PFU, about 10⁸ PFU, about 2*10⁸ PFU, about 5*10⁸ PFU, about 10⁹ PFU, about 2*10⁹ PFU, about 5*10⁹ PFU, about 10¹⁰ PFU, about 2*10¹⁰ PFU, about 5*10¹⁰ PFU, about 10¹¹ PFU, about 2*10¹¹ PFU, about 5*10¹¹ PFU, about 10¹² PFU, about 2*10¹² PFU, about 5*10¹² PFU, about 10¹³ PFU, about 2*10¹³ PFU, about 5*10¹³ PFU, or about 10¹⁴ PFU). In certain of these embodiments, the modified oncolytic virus and the pharmaceutical composition is administered at a dosage of about 10¹¹ PFU or less. In certain of these embodiments, the dosage is 5*10¹⁰ PFU or less, 2*10¹⁰ PFU or less, 5*10⁹ PFU or less, 4*10⁹ PFU or less, 3*10⁹ PFU or less, 2*10⁹ PFU or less, or 10⁹ PFU or less. A particular dosage can be divided and administered multiple times separated by interval, e.g., once every day, twice or more every day, twice or more every month, once every week, once every two weeks, once every three weeks, once a month or once every two months or more. In certain embodiments, the administered dosage may vary over the course of treatment. For example, in certain embodiments, the initially administered dosage can be higher than subsequently administered dosages. In certain embodiments, the administered dosages are adjusted in the course of treatment depending on the response of the administration subject.

The term “PFU” as used herein refers to plaque-forming unit, which is a measure of the number of particles capable of forming plaques.

Dosage regimens may be adjusted to provide the optimum desired response (e.g., a therapeutic response). For example, a single dose may be administered, or several divided doses may be administered over time.

Combination

In certain embodiments, the pharmaceutical compositions may be used in combination with one or more other drugs. In certain embodiments, the composition comprises at least one other drug.

In certain embodiments, the other drugs are anti-tumor agent. Any agents known to be active against tumor may be used as anti-tumor agent. In certain embodiments, the anti-tumor agent is selected from the group consisting of a chemical agent, a polynucleotide, a peptide, a protein, or any combination thereof.

In certain embodiments, the anti-tumor agent is a chemical agent. Illustrative examples of anti-tumor chemical agent include, without limitation, Mitomycin C, Daunorubicin, Doxorubicin, Etoposide, Tamoxifen, Paclitaxel, Vincristine, and Rapamycin.

In certain embodiments, the anti-tumor agent is a polynucleotide. Illustrative examples of anti-tumor polynucleotide include, without limitation, anti-sense oligonucleotides such as bcl-2 antisense oligonucleotides, clusterin antisense oligonucleotides, and c-myc antisense oligonucleotides; and RNAs capable of RNA interference (including small interfering RNA (siRNA), short hairpin RNA (shRNAs), and micro interfering RNAs (miRNA)), such as anti-VEGF siRNA, shRNA, or miRNA, anti-bcl-2 siRNA, shRNA, or miRNA, and anti-claudin-3 siRNA, shRNA, or miRNA.

In certain embodiments, the anti-tumor agent is a peptide or protein. Illustrative examples of anti-tumor peptide or protein include, without limitation, antibodies such as, Trastuzumab, Rituximab, Edrecolomab, Alemtuzumab, Daclizumab, Nimotuzumab, Gemtuzumab, Ibritumomab, and Edrecolomab, protein therapeutics such as, Endostatin, Angiostatin K1-3, Leuprolide, Sex hormone-binding globulin, and Bikunin.

Medical Usage

In another aspect, the present disclosure provides use of the modified oncolytic virus of the present disclosure or the pharmaceutical composition of the present disclosure in the manufacture of a medicament for treating a tumor.

In another aspect, the present disclosure provides the modified oncolytic virus of the present disclosure or the pharmaceutical composition of the present disclosure for use in treating a tumor.

### EXAMPLES

The following Examples are set forth to aid in the understanding of the present disclosure, and should not be construed to limit in any way the scope of the invention as defined in the claims which follow thereafter.

### Example 1: Virus Construction

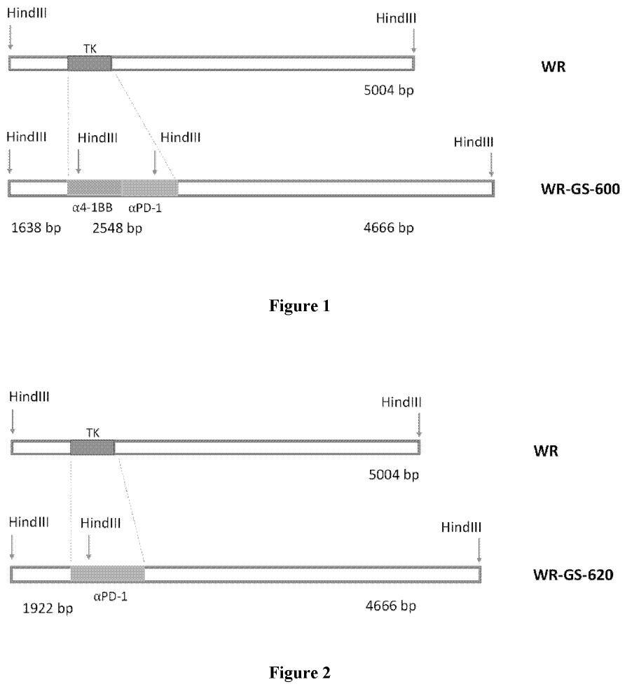

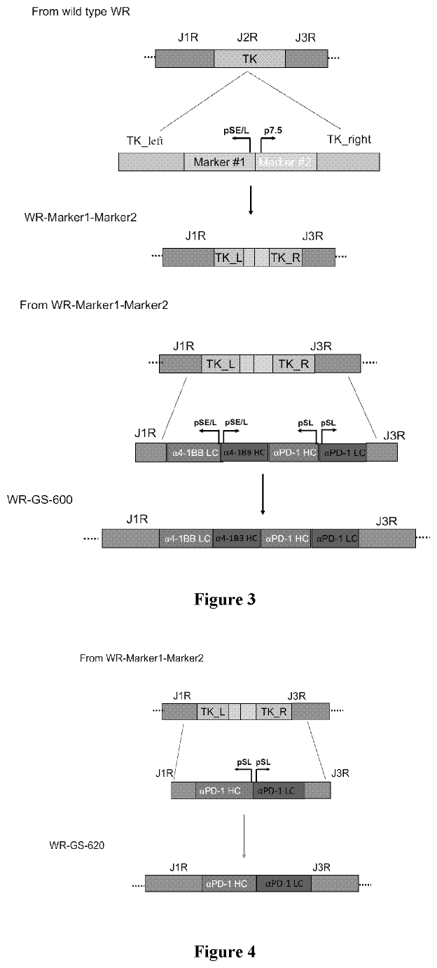

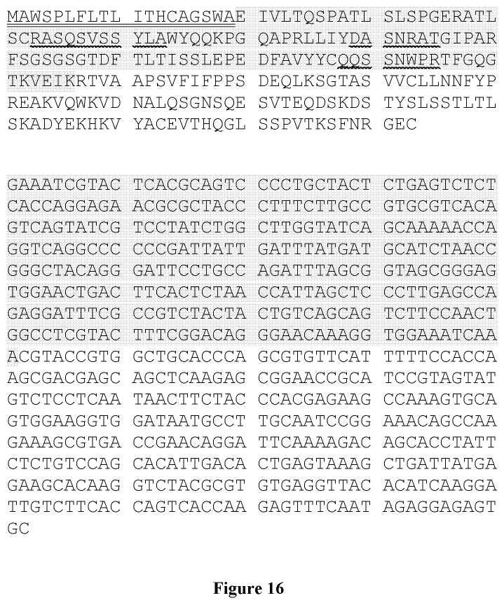

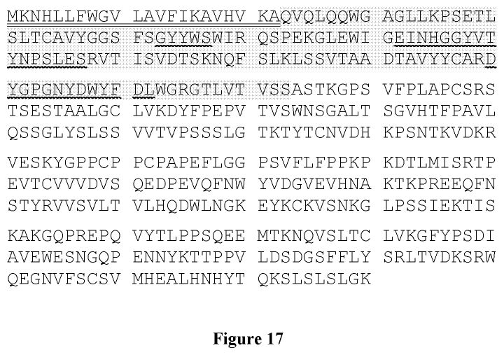

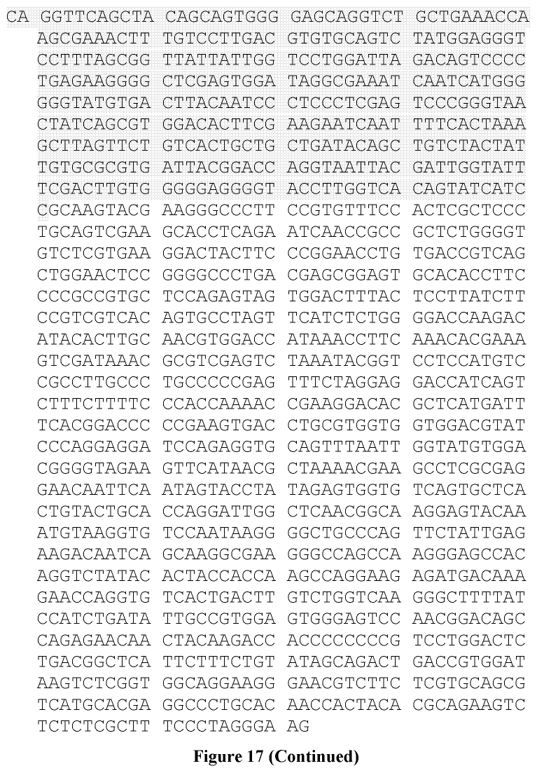

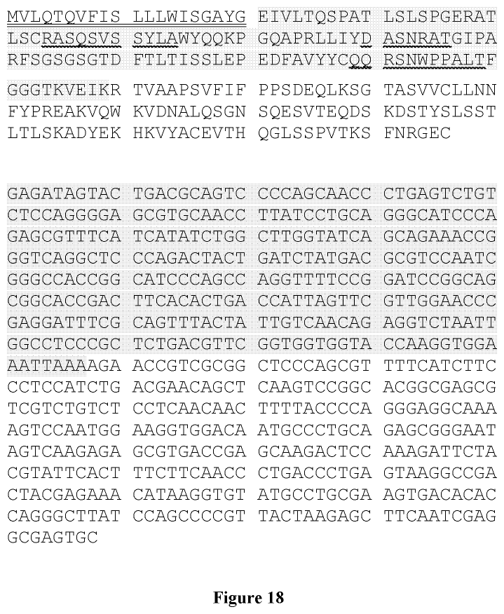

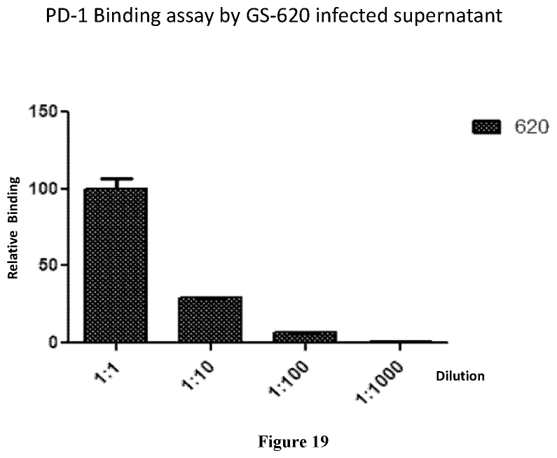

The starting WR strain of vaccinia virus was obtained from ATCC (www.atcc.org: VR-1354). Due to multiple genes involved, WR-GS-600 has been built in a step-by-step engineering approach. In brief, in the first step, WR DNA is recombined with a modified pSEM-1 vector (Rintoul et al., 2011) to insert marker/selection genes into the TK locus. This allows for easy distinction from the wild-type parent for further engineering. Afterwards, a recombination plasmid with flanking sequences of J1R and J3R and encoding anti-human PD-1 (the amino acid sequence of the anti-human PD-1 and the nucleic acid sequence encoding the anti-human PD-1 are shown in FIGS. 15 and 16, respectively) and anti-human 4-1BB (the amino acid sequence of the anti-human 4-1BB and the nucleic acid sequence encoding the anti-human 4-1BB are shown in FIGS. 17 and 18, respectively) was transfected into U2OS cells infected with WR to completely delete TK and insert the antibody sequences. FIG. 1 shows the structure of thymidine kinase (TK) deletion, anti-PD-1 and anti-4-1BB antibodies insertion in WR-GS-600 and FIG. 3 shows schematic diagram of the recombination steps for generating the WR-GS-600.

The recombination reaction was conducted using U2OS cells from a characterized master working cell bank. Three rounds of plaque purification was carried out using U2OS cells and one round using HeLa cells. Afterwards, a filtration step using 0.65 μm filter was incorporated to ensure the final plaques picked were clonal. The detailed information is described in the Table 1 below.

TABLE 1
WR-GS-600 Plaque Generation and Purification
Procedure

| Stage | Material |
|---|---|
| Recombination using U2OS | WR strain, U2OS cells, |
| | recombination plasmids |
| Plaque purification using U2OS | U2OS cells |
| Plaque Purification using U2OS | |
| with CMC overlay | |
| Plaque purification with CMC overlay | |
| and filtration of 0.65 μm filter | |
| Amplification of Material | 4 roller bottles of HeLa cells |
| | were used to amplify the |
| | final two clones to get enough |
| | material for following assays |

After further plaque purification, antibody expression was monitored by immunofluorescence and flow cytometry. U2OS and HeLa cells were mock-infected (i.e. infected with a control solution without virus), infected with a control virus with antibody expression or with purified clones of WR-GS-600, separately.

Finally one unique clone with verified DNA sequence and high level of antibody expression was amplified in two roller bottles (1700 cm²). Cells were pelleted and then resuspended in 1 mM Tris pH 9.0. After one round of freeze-thaw (−80/37 degree), the mixture was pelleted again. The supernatant was aliquot into 12 cryogenic tubes in 1 ml each (pre-Master Virus Bank). The pelleted cells were resuspended in 3 ml of 1 mM Tris pH 9.0 and underwent another round of freeze/thaw. The supernatant was collected after pelleting, and then underwent overnight benzonase treatment, and sucrose purification. Titers for pre-MVB and benzonase purified were determined using U2OS cells. Titers have been found to be in the range of 1.0-2.1*10⁹ pfu/mL for a total 5 mL stock, which is similar to that of the parental WR virus.

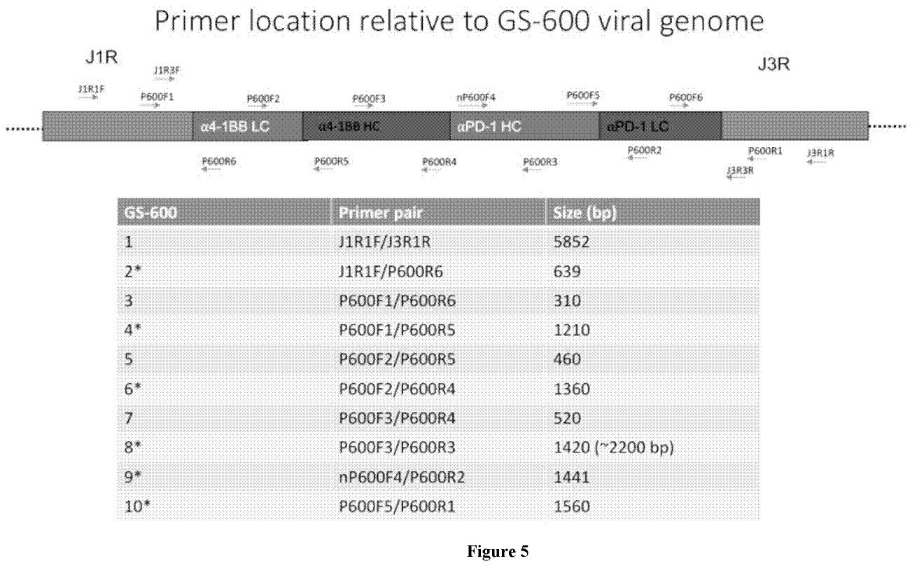

WR-GS-610 (inserted a gene encoding anti-human 4-1BB) and WR-GS-620 (inserted a gene encoding anti-human PD-1) were manufactured by the same protocol as WR-GS-600, excepted for that WR-GS-600 was inserted by both the gene encoding anti-human PD-1 and the gene encoding anti-human 4-1BB. FIG. 2 shows the structure of TK deletion and anti-4-1BB antibody insertion in WR-GS-620 and FIG. 4 shows schematic diagram of the recombination step for WR-GS-620.

### Example 2: Characterization of WR-GS-600, WR-GS-610 and WR-GS-620

During the engineering process of these new viruses, their genomic integrity and protein expression were closely monitored.

PCR, Sequencing and Restriction Digestion

Genomic DNA of the viruses was isolated from sucrose cushion purified by treating virus preparations with Benzonase endonuclease, pelleting through sucrose, followed by Proteinase K and detergent treatment, then DNA was extracted and recovered using phenol/chloroform/isoamyl alcohol extraction, and ethanol precipitation.

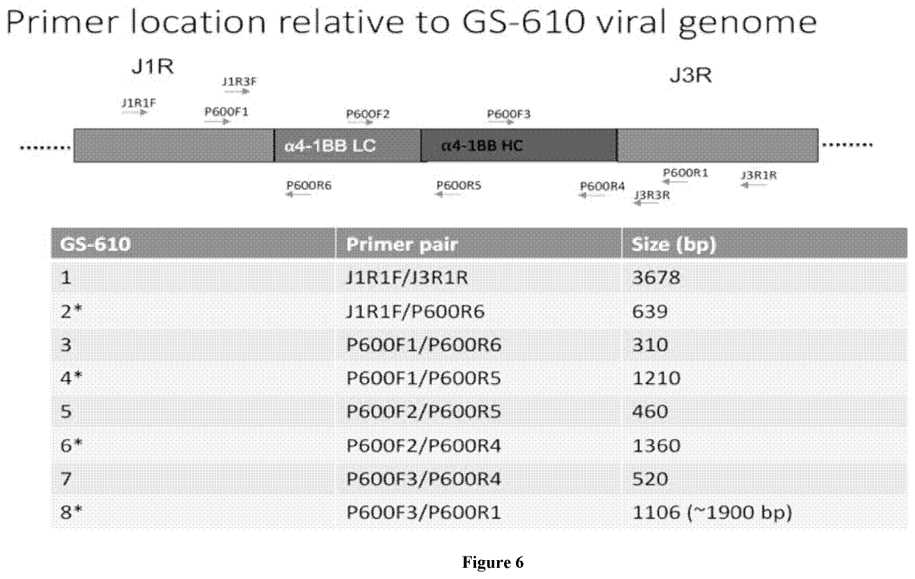

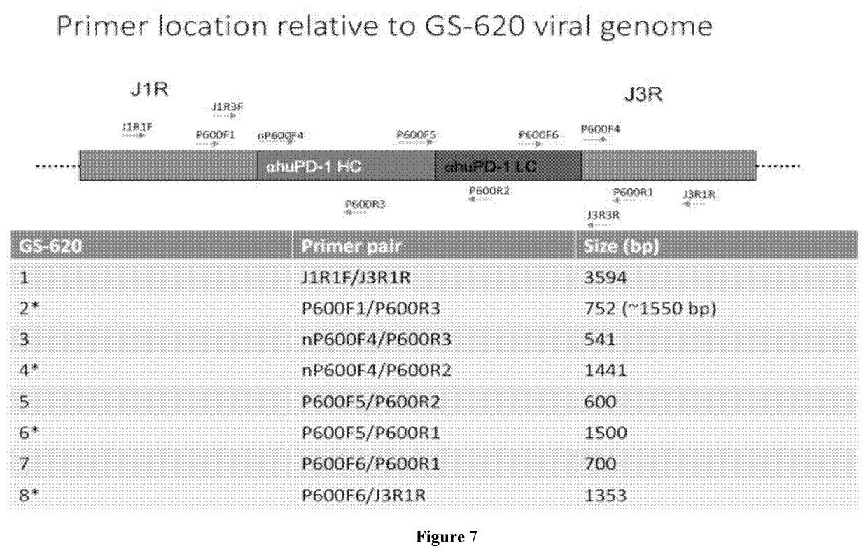

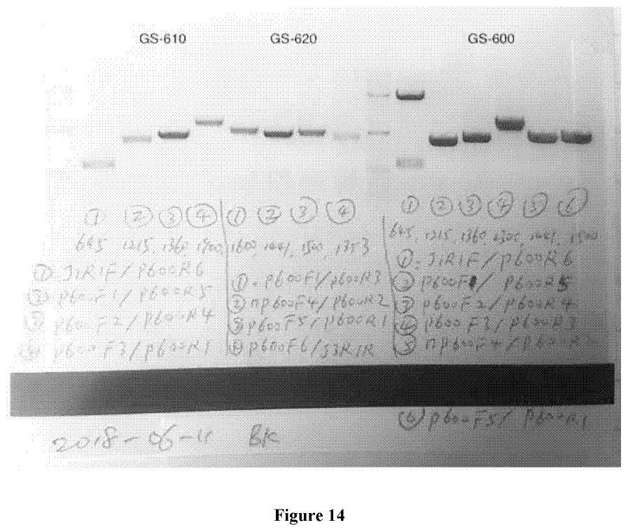

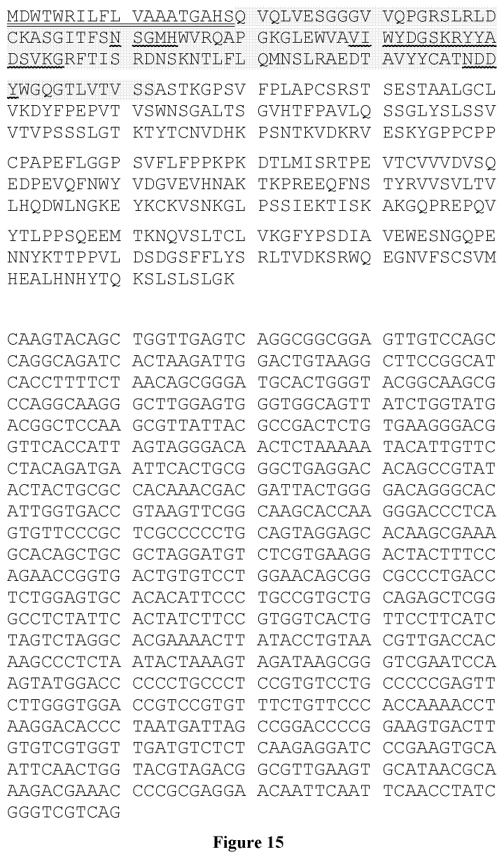

In order to ensure the viral genome has the expected sequences harbouring the designed antibody sequences, a series of primers have been designed, including those within the recombination regions and those outside the engineering sections. Viruses (WR-GS-600, WR-GS-610 and WR-GS-620) identity were confirmed by qPCR (TaqMan). The primers used in PCR are shown in Table 2. The locations of the primers in the viral genomes are shown in FIGS. 5 to 7, wherein the predicted size of PCR bands are depicted in the FIGS. 5 to 7. The result of PCR amplification of genomic DNA of WR-GS-600, WR-GS-610 and WR-GS-620 is show in FIG. 14.

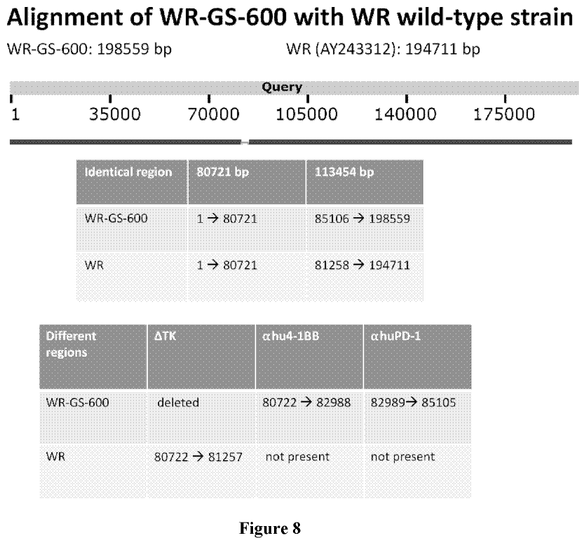

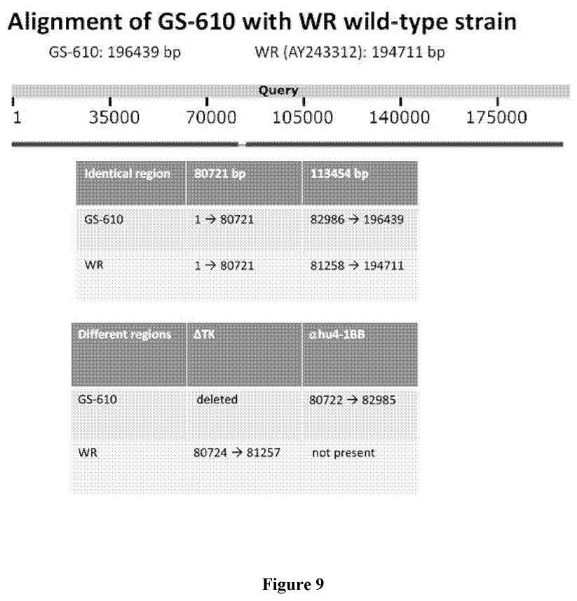

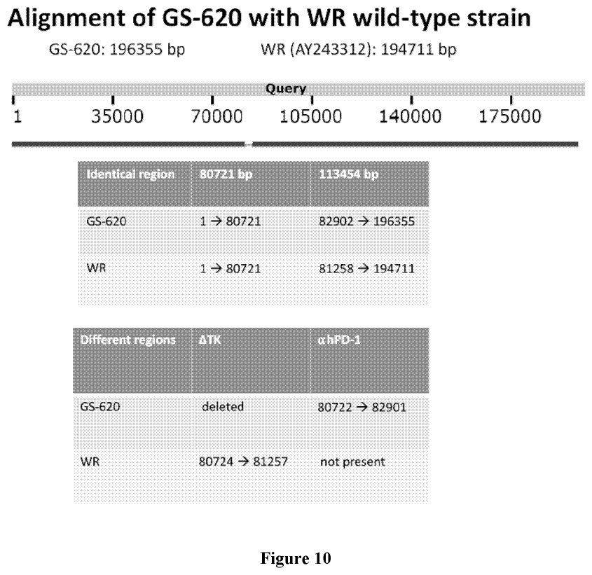

TK deletion was also verified through Sanger sequencing. FIGS. 8, 9 and 10 show the genetic changes from WR after insertion of antibody encoding genes. Alignment of Sanger sequencing of WR-GS-600 viral genome against designed DNA sequences for expressing anti-hu4-1BB and anti-huPD-1 in WR-GS-600 was conducted. The Alignment showed that the viral genome of WR-GS-600 is identical to the designed DNA sequence.

TABLE 2
Primers Used for PCR and Sequencing

| | | Alignment | Alignment | Alignment | Alignment |
|---|---|---|---|---|---|
| Primer | Sequence | with GS-600 | GS-610 | GS-620 | with WR |
| J1R1F | ATGGATCACA | 80247-80276 | 80247- | 80247- | 80247- |
| | ACCAGTATCT | | 80276 | 80276 | 80276 |
| | CTTAACGATG | | | | |
| J3R1R | GAAATATAGA | 86098-86069 | 83924- | 83840- | 82196- |
| | TTGTTGTAGA | | 83895 | 83811 | 82167 |
| | AATAGTACCT | | | | |
| J1R3F | ATATCGCATT | 80650-80673 | 80650- | 80650- | 80650- |
| | TTCTAACGTG | | 80673 | 80673 | 80673 |
| | ATGG | | | | |
| J3R3R | GGTTTATCTA | 85250-85227 | 83076- | 82992- | 81348- |
| | ACGACACAAC | | 83053 | 82969 | 81325 |
| | ATCC | | | | |
| P600F1 | GATGCGATTC | 80576-80599 | 80576- | 80576- | N/A |
| | AAAAAAGAA | | 80599 | 80599 | |
| | TCCTC | | | | |
| P600F2 | GGATAAGGTT | 81326-81349 | 81326- | N/A | N/A |
| | GCACGCTCCC | | 81349 | | |
| | CTGG | | | | |
| P600F3 | CTTTACTCCT | 82166-82189 | 82166- | N/A | N/A |
| | TATCTTCCGT | | 82189 | | |
| | CGTC | | | | |
| P600F4 | GCAACGCTTC | 83045-83068 | N/A | 80787- | N/A |
| | GTGCATCACG | | | 80810 | |
| | GAGC | | | | |
| P600F5 | GTAGTCCTTC | 83886-83909 | N/A | 81628- | N/A |
| | ACGAGACATC | | | 81651 | |
| | CTAG | | | | |
| P600F6 | GCCGTCTACT | 84746-84769 | N/A | 82488- | N/A |
| | ACTGTCAGCA | | | 82511 | |
| | GTCT | | | | |
| P600R1 | TGTGTACCGG | 85445-85421 | 83271- | 83187- | N/A |
| | GAGCAGATCC | | 83247 | 83163 | |
| | TATAT | | | | |
| P600R2 | CGGCGCAGTG | 84485-84462 | N/A | 82227- | N/A |
| | AGTAATCAAG | | | 82204 | |
| | GTCA | | | | |
| P600R3 | ATTAGCCGGA | 83585-83562 | N/A | 81327- | N/A |
| | CCCCGGAAGT | | | 81304 | |
| | GACT | | | | |
| P600R4 | GGCTTGGTGG | 82685-82662 | 82685- | N/A | N/A |
| | TAGTGTATAG | | 82662 | | |
| | ACCT | | | | |
| P600R5 | ACCCCCCATG | 81785-81762 | 81785- | N/A | N/A |
| | ATTGATTTCG | | 81762 | | |
| | CCTA | | | | |
| P600R6 | CTCCAAAGAT | 80885-80862 | 80885- | N/A | N/A |
| | TCTACGTATT | | 80862 | | |
| | CACT | | | | |

Restriction enzyme HindIII cut around the TK region of WR and produced a band of 5004 bp. When TK is deleted and anti-huPD-1 and/or anti-hu4-1BB antibodies inserted in WR-GS-600, two extra HindIII restriction sites were introduced, which leads to three bands at 1638, 2548, and 4666 bp, respectively. For WR-GS-620, the wild type WR's 5004 bp band was replaced with two bands at 1922 and 4666 bp. These different in restriction digestion patterns can be employed for quick identification of these viruses. The results are shown in Table 3.

TABLE 3
HindIII digestion of viral genomes

| Virus | Wester Reserve | | WR-GS-600 | | WR-GS-620 | |
|---|---|---|---|---|---|---|
| Segments | Ends | Size (bp) | Ends | Size (bp) | Ends | Size (bp) |
| 1 | 109902-163533 | 53632 | 113750-167381 | 53632 | 111486-165117 | 53632 |
| 2 | 163534-194711 | 31178 | 167382-198559 | 31178 | 165118-196295 | 31178 |
| 3 | 1-22092 | 22092 | 1-22092 | 22092 | 1-22092 | 22092 |
| 4 | 93846-109901 | 16056 | 97694-113749 | 16056 | 95430-111485 | 16056 |
| 5 | 43824-59029 | 15206 | 43824-59029 | 15206 | 43824-59029 | 15206 |
| 6 | 30407-43823 | 13417 | 30407-43823 | 13417 | 30407-43823 | 13417 |
| 7 | 67237-76113 | 8877 | 67237-76113 | 8877 | 67237-76113 | 8877 |
| 8 | 85233-93845 | 8613 | 89081-97693 | 8613 | 86817-95429 | 8613 |
| 9 | 60745-67236 | 6492 | 60745-67236 | 6492 | 60745-67236 | 6492 |
| 10 | 80229-85232 | 5004 | 84415-89080 | 4666 | 82151-86816 | 4666 |
| 11 | 25877-30406 | 4530 | 25877-30406 | 4530 | 25877-30406 | 4530 |
| 12 | 76114-80228 | 4115 | 76114-80228 | 4115 | 76114-80228 | 4115 |
| 13 | | | 81867-84414 | 2548 | | |
| 14 | 23655-25876 | 2222 | 23655-25876 | 2222 | 23655-25876 | 2222 |
| 15 | | | 80229-81866 | 1638 | 80229-82150 | 1922 |
| 16 | 22093-23654 | 1562 | 22093-23654 | 1562 | 22093-23654 | 1562 |
| 17 | 59303-60744 | 1442 | 59303-60744 | 1442 | 59303-60744 | 1442 |
| 18 | 59030-59302 | 273 | 59030-59302 | 273 | 59030-59302 | 273 |

Immunofluorescence

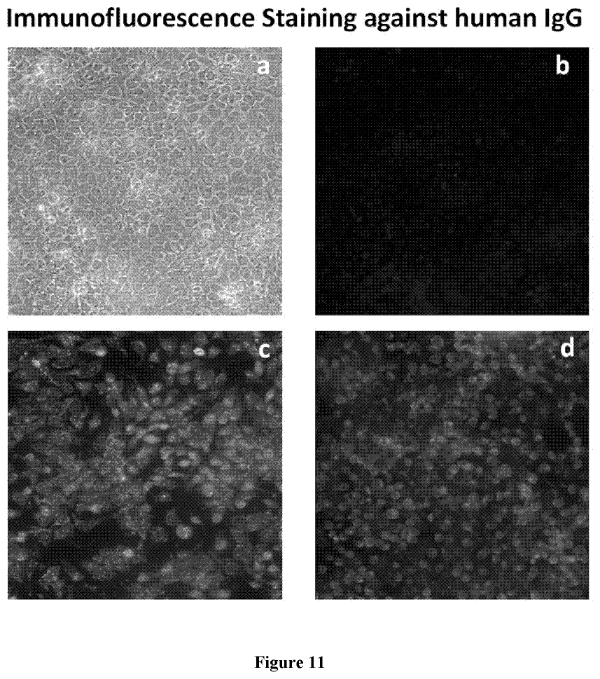

Transgene expression of the human antibodies was verified via immunofluorescence against human IgG (see FIG. 11). FITC-conjugated goat anti-human IgG (H+L) (Invitrogen, Cat #62-8411) was used to stain viral infected U2OS cells.

Flow Cytometry Analysis

Flow cytometry analysis of HeLa cells mock-infected, infected with a control WR virus (WR-mCherry), infected with WR-GS-600, and infected with WR-GS-620, separately, confirmed the specific expression of the human antibodies when infected by WR-GS-600 and WR-GS-620. Detected human IgG from the infected supernatants in Western blot provided further evidence of the expression of the human antibodies.

Western Blotting

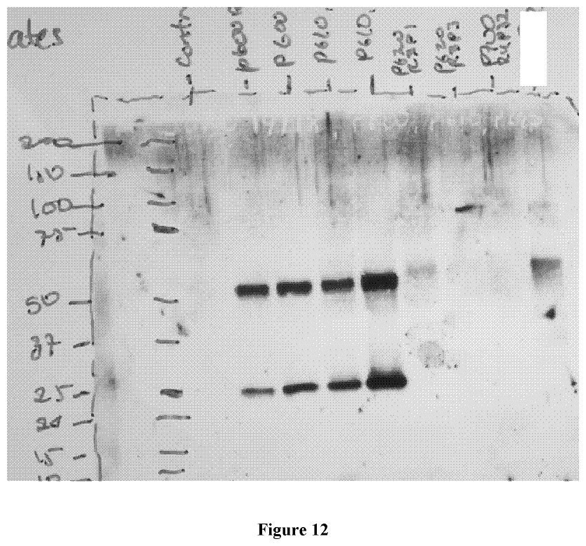

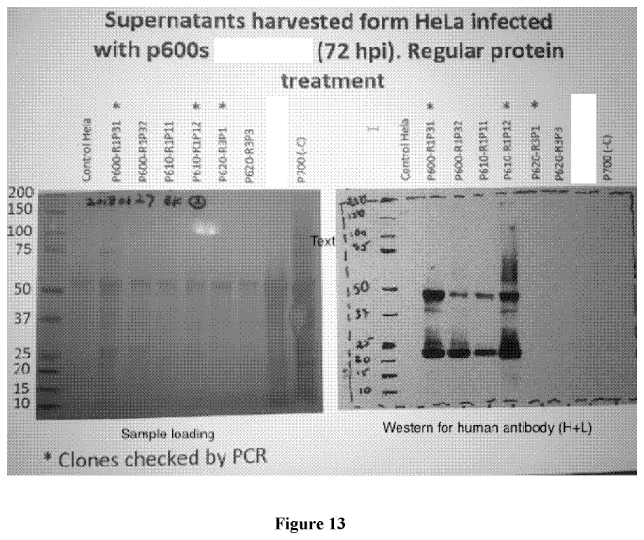

Western blot detecting human IgG from the infected supernatants provided further evidence of antibody expression. FIGS. 12 and 13 shows expression of anti-PD-1 and anti-41-BB antibodies by recombinant viruses (WR-GS-600, WR-GS-610 and WR-GS-620) using Western blotting, where cell lysates and supernatants were used, respectively.

Functional Characterization of Expressed Anti-PD-1 Antibody Using PD-1 Binding ELISA

Recombinant Human PD-1 Fc chimera (R&D Systems, Minneapolis, Minn.) is resuspended with Dulbecco's Phosphate Buffered Saline (DPBS) containing 0.1% bovine serum albumin (BSA) to 0.2 mg/ml and diluted with DPBS to a final concentration of 0.03 μg/ml. Nunc-Immuno Maxisorp 96 well plates are coated with 0.1 ml per well of the recombinant PD-1 Fc chimera leaving empty wells for nonspecific binding controls and incubated at 4° C. overnight. The coating solution is removed and plates washed with wash buffer (0.05% Tween-20 in DPBS, 200 μL per well each time). Blocking buffer (5% non-fat dry milk, 0.05% Tween-20 in DPBS, 200 μL per well each time) is added to all wells and incubated at 4° C. for 1 hour with mixing. The blocking buffer is removed and plates are washed with wash buffer. Serial dilutions of WR-GS-620 and WR-GS-600 supernatants are prepared in DPBS and diluted supernatant (100 μL per well) is added to the plates. Plates are incubated for 1.5 hours at room temperature. Antibody containing supernatant solution is removed and the plates are washed with wash buffer. Horseradish peroxidase labeled goat anti-human IgG, F(ab′)₂ specific F(ab′)₂ antibody (Jackson Immunoresearch, West Grove, Pa.) is diluted with DPBS and 100 μL per well added to the plates. The plates are incubated for 1 hour at room temperature and washed with wash buffer. 100 μL per well SureBlue TMB microwell peroxidase substrate (Kirkegaard & Perry Labs Gaithersburg, Md.) is added and incubated for 20 minutes at room temperature. The reaction is stopped by the addition of an equal volume of 2M H₂SO₄ and absorbance is read at 450 nm on a Molecular Devices Spectra Max 340 (Molecular Devices, Sunnyvale, Calif.).

Supernatants from U2OS infected with MOI 0.05 for 48 hours were used for the analysis and results are shown in FIG. 19. These results suggest that the expressed anti-PD-1 antibody of GS-620 can specifically bind to PD-1 and the binding is concentration dependent.

Functional Characterization of Expressed Anti-4-1BB Antibody Using 4-1BB Binding ELISA

Human 4-1BB IgG1Fc chimera (R&D Systems, Minneapolis, Minn.) is resuspended with Dulbecco's Phosphate Buffered Saline (DPBS) containing 0.1% bovine serum albumin (BSA) to 0.2 mg/ml and diluted with DPBS to a final concentration of 0.03 μg/ml. Nunc-Immuno Maxisorp 96 well plates are coated with 0.1 ml per well of the recombinant 4-1BB chimera leaving empty wells for nonspecific binding controls and incubated at 4° C. overnight. The 4-1BB solution is removed and plates are washed with wash buffer (0.05% Tween-20 in DPBS). Blocking buffer (5% non-fat dry milk, 0.05% Tween-20 in DPBS) is added to all wells and incubated at 4° C. for 1 hour with mixing. The blocking buffer is removed and plates are washed with wash buffer. Serial dilutions of WR-GS-610 and WR-GS-600 supernatants are prepared in DPBS and diluted supernatant is added to the plates. Plates are incubated for 1.5 hours at room temperature. Antibody containing supernatant solution is removed and the plates are washed with wash buffer. Horseradish peroxidase labeled goat anti-human IgG, F(ab′)₂ specific F(ab′)₂ antibody (Jackson Immunoresearch, West Grove, Pa.) is diluted with DPBS and added to the plates. The plates are incubated for 1 hour at room temperature and washed with wash buffer. SureBlue TMB microwell peroxidase substrate (Kirkegaard & Perry Labs Gaithersburg, Md.) is added and incubated for 20 minutes at room temperature. The reaction is stopped by the addition of an equal volume of 2M H₂SO₄ and absorbance is read at 450 nm on a Molecular Devices Spectra Max 340 (Molecular Devices, Sunnyvale, Calif.).

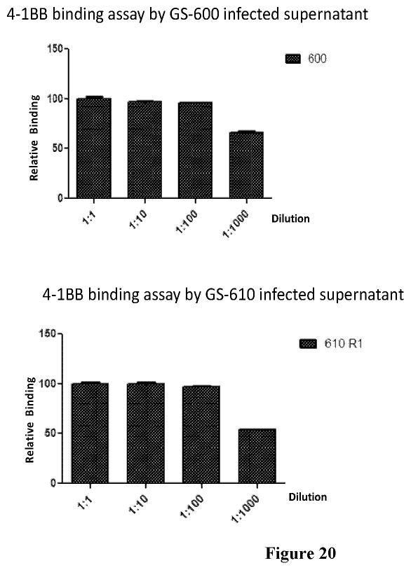

Supernatants from U2OS infected with MOI 0.05 for 48 hours were used for the analysis and results are shown in FIG. 20. These results suggest that the expressed anti-4-1BB antibody of GS-600 and GS-610 can specifically bind to 4-1BB and the binding is concentration dependent.

The above tests show that WR-GS-600, WR-GS-610 and WR-GS-620 were well constructed and functional corresponding antibodies (anti-PD1 antibody for WR-GS-600 and WR-GS-620, and anti-4-1BB antibody for WR-GS-600 and WR-GS-610) can be expressed.

### Example 3: In Vivo Study of WR-GS-600, WR-GS-610 and WR-GS-620 Recombination Viruses

The following studies are conducted to determine if WR-GS-600, WR-GS-610 and WR-GS-620 recombination viruses are safe to mice and whether the recombinant virus can target and penetrate tumors in mice. The delivery route can be intravenously (IV) or intraperitoneally (IP). All animal experiments were conducted following the guidance of local animal care committee.

Measurement of Cytotoxicity (Cell Killing Data) of WR-GS-600, WR-GS-610 and WR-GS-620 in CT26, MC38, HT-29 and HCT-116 Cell Lines

Colorectal cancer cell lines CT26-LacZ (murine), MC38-Luc (murine), HT-29-Luc (human) and HCT-116-Luc (human) were used for in vitro cytotoxicity testing. WR-GS-600, WR-GS-610 and WR-GS-620 were prepared in three different MOIs respectively, i.e. 0.01 MOIs (3E2 PFU), 0.1 MOIs (3E3 PFU) and 1.0 MOIs (3E4 PFU). Measurements were carried out at three different time points, i.e. 24 hours, 48 hours and 72 hours.

Cell preparation: each cell line was first plated in two 15 cm tissue culture dishes and incubated until sub-confluent between 75-90%. Cells were washed and counted using conventional methods known to a person having ordinary skill in the art. Each well of a 96-well flat bottom plate was seeded with about 3E4 cells. Each cell type requires 9 plates for 9 different experimental conditions.

Virus dilution preparation: viruses were thawed on ice followed by being thawed in 37° C. water bath to ensure complete defrost. Thawed viruses were subjected to vortex at a maximum speed twice and each time for 20 seconds. WR-GS-600, WR-GS-610 and WR-GS-620 viruses were prepared at three different concentrations, i.e. MOI 1.0, MOI 0.1 and MOI 0.01. Viruses of 50 μL were added to corresponding wells followed by rocking 96-well flat bottom plates gently in 4 quadrants for mixture. Plates were incubated at 37° C. supplemented with 5% CO₂. MOI 1.0 corresponds to 3E4 PFU/50 μL or 600 PFU/μL or 6E5 PFU/mL. MOI 0.1 corresponds to 3E3 PFU/50 μL or 60 PFU/μL or 6E4 PFU/mL. MOI 0.01 corresponds to 3E2 PFU/50 μL or 6 PFU/μL or 6E3 PFU/mL.

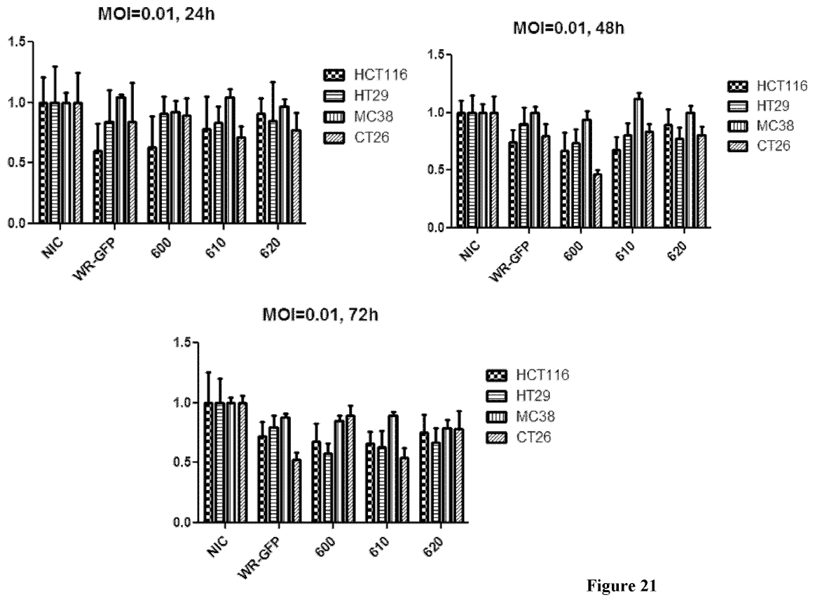

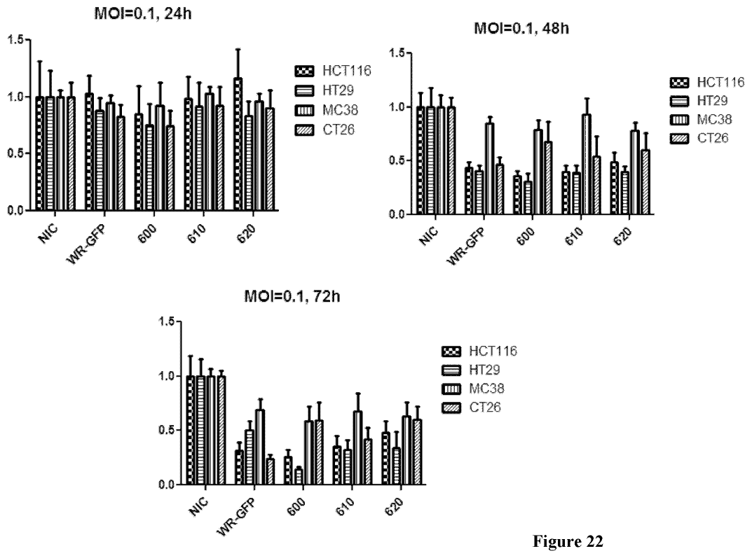

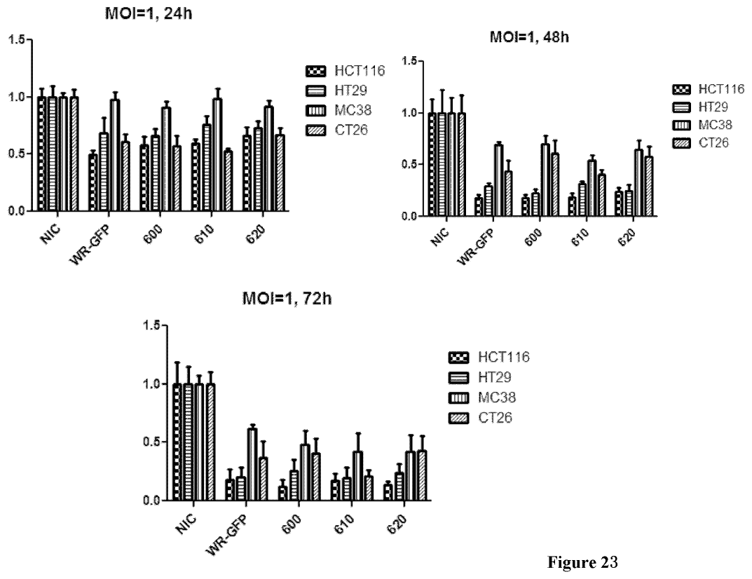

Alamar Blue was used to detect the cytotoxicity of the viruses in the above-mentioned four cell lines using conventional methods known to a person having ordinary skill in the art. Cell viability was calculated with 6 replicates for each condition. FIGS. 21-23 show that there is no significant differences in the cell viability of the above-mentioned four cell types with treatment of WR, WR-GS-600, WR-GS-610 and WR-GS-620 at three different concentrations and three different time points. This suggests that incorporation of polynucleotide sequences for checkpoint inhibitor antibodies into vaccinia virus (WR) genome does not alter the cytotoxicity nature of the viruses. FIGS. 21-23 further show that cell viability of HT-29 and HCT-116 cell lines decreased more significant than that of CT-26 and MC-38 upon treatment of viruses, indicating that human cancer cells are more sensitive to viral infection and killing.

Measurement of Bio-Distribution of Viral Vectors

Tissue Homogenization:

25 Balb/C mice (Jackson Lab) in 5 different groups were sacrificed one at a time. After disinfected spray, mice were opened up, from which 50 to 100 mg of either tumor, lung, spleen, liver, brain or ovary were excised. The remaining tissues were snap frozen with OCT. Excised tissues were weighted and placed into 2.0 mL Eppendorf tubes. Tissue samples were frozen overnight at −80° C. The tissue samples were homogenized the following day in a manner known to a person having ordinary skill in the art. Briefly, two autoclaved 5 mm TissueLyser beads were dispensed into each tube. In total, 48 tubes were loaded into TissueLyser. Homogenization was conducted at 28 Hz for 1 minute. Following this, the insert of the adaptor was turned 180° and homogenization was run for another 1 minute to achieve uniform homogenization. After this, 500 μL of DMEM was added to each sample. Tubes were centrifuged at 3500 g for 2 minutes. Supernatants were transferred to 1.5 mL Eppendorf tubes and stored at −80° C. before titer determination.

24-Well Format for Titer Determination:

U2OS cells were used for viral titer determination, and 10E2 PFU/mL JX594 stock (a) and 31.0 PFU/mL JX594 stock (b) were prepared and used as positive controls.

For 5 tissues, i.e. brain (B), liver (V), lung (L), Ovary (O) and Spleen (S), three concentrations were prepared for each virus of WR-GS-600, WR-GS-610 and WR-GS-620: (1) straight 150 μL for infection; (2) 98 μL from (1) in 212 μL DMEM, mix, take 150 μL for infection; and (3) 98 μL from (2) in 212 μL DMEM, mix, take 150 μL for infection.

For controls (C), U2OS cells were treated with (1) 150 μL of 10E2 PFU/mL JX594 stock (a); (2) 150 μL of 31.0 PFU/mL JX594 stock (b); or (3) 150 μL of DMEM.

For tumor (T), six concentrations were prepared for each virus of WR-GS-600, WR-GS-610 and WR-GS-620: (1) straight 150 μL for infection; (2) 98 μL from (1) in 212 μL DMEM, mix, take 150 μL for infection; (3) 98 μL from (2) in 212 μL DMEM, mix, take 150 μL for infection; (4) 98 μL from (3) in 212 μL DMEM, mix, take 150 μL for infection; (5) 98 μL from (4) in 212 μL DMEM, mix, take 150 μL for infection; and (6) 98 μL from (5) in 212 μL DMEM, mix, take 150 μL for infection.

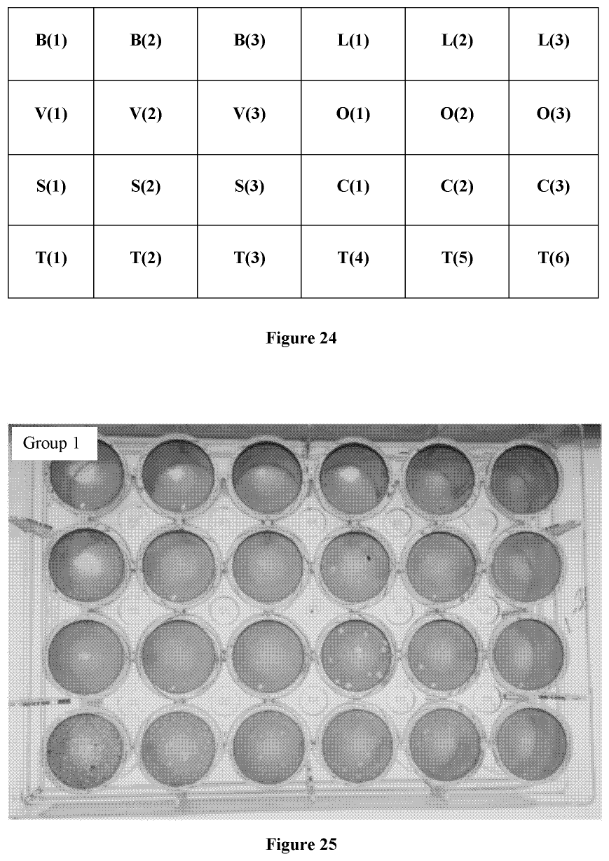

A 24-well plate was set up as shown in FIG. 24. Each plate was seeded with tumor, lung, spleen, liver, brain and ovary cells prepared using the methods as described in the section Tissue homogenization from one mouse.

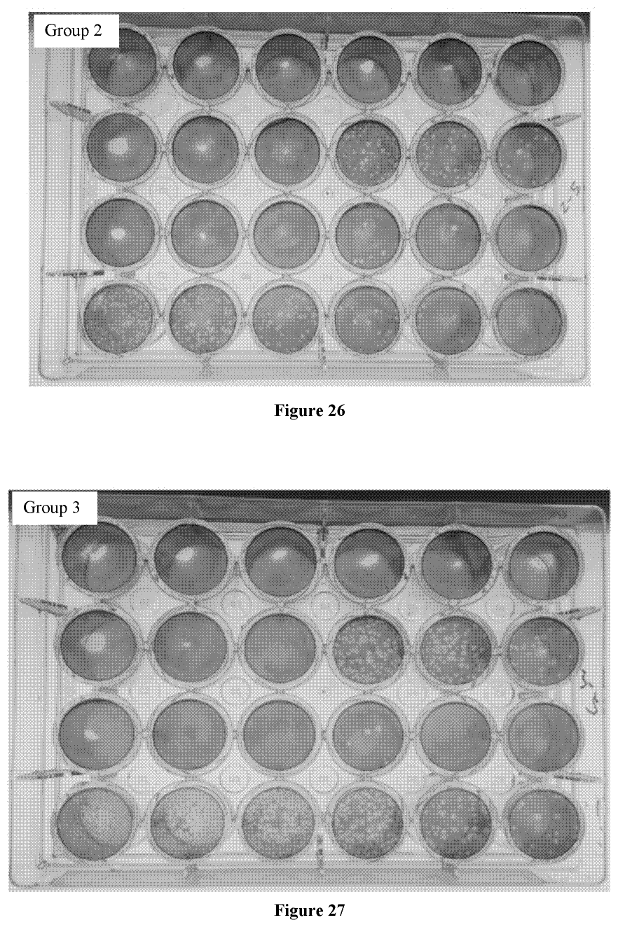

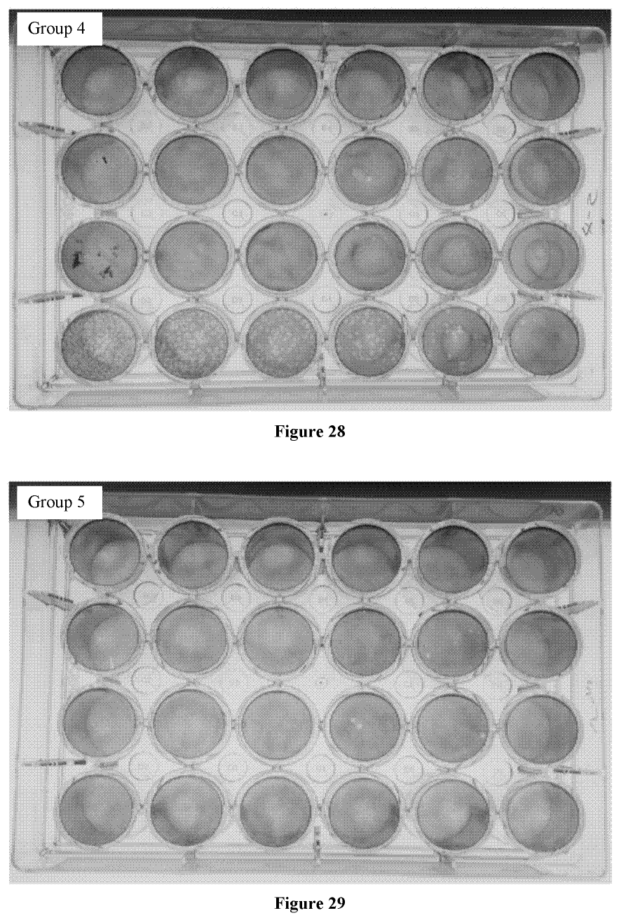

As mentioned above, 25 mice were divided into 5 groups, each group having 5 mice, i.e. 5 plates. Tumor, lung, spleen, liver, brain and ovary cells in Group 1 were infected with WR-GS-610, in Group 2 were infected with WR, in Group 3 were infected with WR-GS-620, in Group 4 were infected with WR-GS-600, and all the cells in Group 5 (except the cells in the positive control wells) were treated with formulation buffer (FB) as a negative control, wherein the formulation buffer comprises 30 mM Tris, 10% sucrose and 150 mM NaCl with a pH value of 7. FIG. 25 shows that WR-GS-610 viral plaques are only present in tumor cell wells. FIG. 26 shows that WR viral plaques are present in both tumor cell wells and ovary cell wells. FIG. 27 shows that WR-GS-620 viral plaques are present in both tumor cell wells and ovary cell wells. FIG. 28 shows that WR-GS-600 viral plaques are present in only tumor cell wells. FIG. 29 shows that there is no viral plaques in tumor cell wells in Group 5. These data suggests that WR-GS-600 and WR-GS-610 can target tumors more specifically as compared to WR-GS-620 and WR.

In Vivo Viral Distribution in Injected Subcutaneous Tumor and Other Tissues:

Goal: Safety and bio-distribution of viral vectors

Study Protocol
i. Order 35 Balb/C mice (Charles River). Mice are distributed across 5 treatment groups, PBS control (FB), and WR, WR-GS-600, WR-GS-610 and WR-GS-620 groups.
ii. Treatment starts when the tumor group's tumor reaching 5 mm in size.
iii. Three injections of viruses via tail vein injection (schedule Day 1, 4, 7)
iv. Monitor mice weight and wellness.
v. At day 9, mice are sacrificed and tissues from brain, lung, liver, ovary, spleen are collected. Vaccinia titers in different tissues are determined by plaque assay on U2OS cells.

Tables 3-5 below summarize the treatment groups, treatment schedule, and anesthesia, endpoints and euthanasia.

TABLE 3
Treatment Groups:

| Group | Group | | | |
|---|---|---|---|---|
| # | Size | 1^{st} Injections | Dose | Schedule |
| 1 | 5 | Control- | n/a | Day 1, 4, 7 |
| | | Formulation Buffer | | |
| 2 | 5 | P600 | 1E7 PFU | Day 1, 4, 7 |
| 3 | 5 | P610 | 1E7 PFU | Day 1, 4, 7 |
| 4 | 5 | P620 | 1E7 PFU | Day 1, 4, 7 |
| 7 | 5 | WR | 1E7 PFU | Day 1, 4, 7 |

TABLE 4
Treatment Schedule:

| Date | Day | Procedure |
|---|---|---|
| 26 Jul. 2019 | −11 | Weigh and Ear Notch |
| 26 Jul. 2019 | | Inject CT26 cells (#) SC right flank |
| 6 Aug. 2019 | 1 | Inject IT 50 μL (1E7 PFU) Virus |
| 9 Aug. 2019 | 4 | Inject IT 50 μL (1E7 PFU) Virus |
| 12 Aug. 2019 | 7 | Inject IT μL (1E7 PFU) Virus |
| 14 Aug. 2019 | 9 | Cardiac bleed, cervical dislocation. Harvest |
| | | spleen, liver, ovary, lung and brain |

TABLE 5
Anesthesia, Endpoints and Euthanasia:

[symbol]

Anesthesia Required Method: Isoflurane
Procedures Requiring Anesthesia: Ear notching,
cardiac bleed, euthanasia
Endpoints:
Weight loss >25%;
M3/severe dehydration despite fluid therapy;
M3/severe neurological signs (circling, spinning, unable
to maintain upright position or move);
M3/severe respiratory distress
Method(s) of euthanasia: Cardiac bleed and cervical dislocation

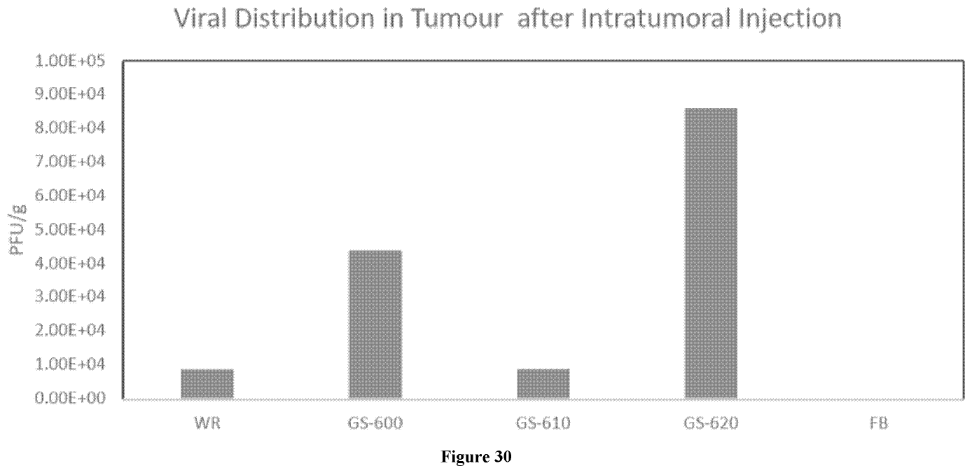

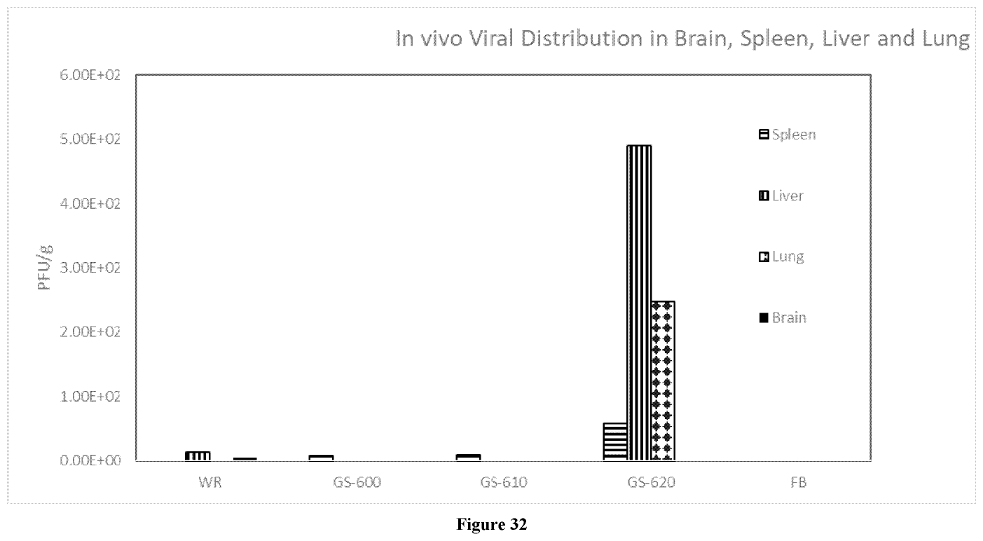

Data were presented by averaging over 5 mice per group. FIGS. 30-32 show that WR-GS-600 and WR-GS-610 are preferably present in tumor and very few WR-GS-600 and WR-GS-610 viruses were observed in ovary, brain, spleen, liver and lung. In contrast, large amount of WR-GS-620 viruses were observed in tumor, ovary, brain, spleen, liver and lung after intratumoral injection. These data confirms that WR-GS-600 and WR-GS-610 have higher tumor targeting specificity than WR-GS-620. Moreover, the second and third bars in the bar graphs of FIGS. 30-32 show that while the numbers of PFU per gram of tissue for WR-GS-600 and WR-GS-610 are similar in ovary (WR-GS-610 is slightly higher than WR-GS-600), brain, spleen, liver and lung, the number of PFU per gram of tumor tissue for WR-GS-600 is about three times higher than that for WR-GS-610. This suggests that tumors can be infected by WR-GS-600 more severely than WR-GS-610 when other tissues are similarly infected by WR-GS-600 and WR-GS-610.

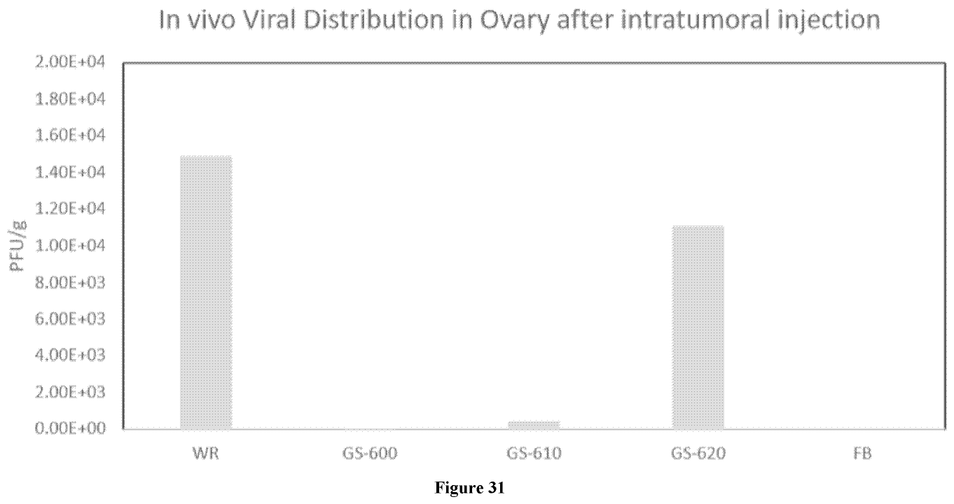

Previous studies have shown that vaccinia Western Reserve strain can infect normal mouse organs, particularly ovary (Zhao Y. et al, Viral Immunology, 2011, 24, 387), which coincides with the results presented in FIG. 31.

Collectively, these data suggest that incorporation of a first heterologous polynucleotide encoding an immune checkpoint inhibitor and a second heterologous polynucleotide encoding an immuno activator into an oncolytic virus, such as WR, results in synergistic effects of tumor targeting and infection, which cannot be otherwise achieved by wild-type oncolytic virus, or modified oncolytic virus having only a first heterologous polynucleotide or only a second heterologous polynucleotide.

Measurement of Tumor Size Change of CT-26 Murine Tumor Model after Different Viral Infection

25 Balb/C mice (Jackson Lab) were implanted with CT26 tumor (CT-26 LacZ 5E6 cells SG right flank). The mice were further distributed across 5 treatment groups: formulation buffer (FB), WR, WR-GS-600, WR-GS-610 and WR-GS-620. Treatment starts when the tumor group's tumor reaching 5 mm in size. Different viruses of 1E7 pfu were injected intratumorally at Day 1, 4 and 7, and mice weight and wellness were monitored. Tumor growth was followed by measuring the tumor size with a caliper.

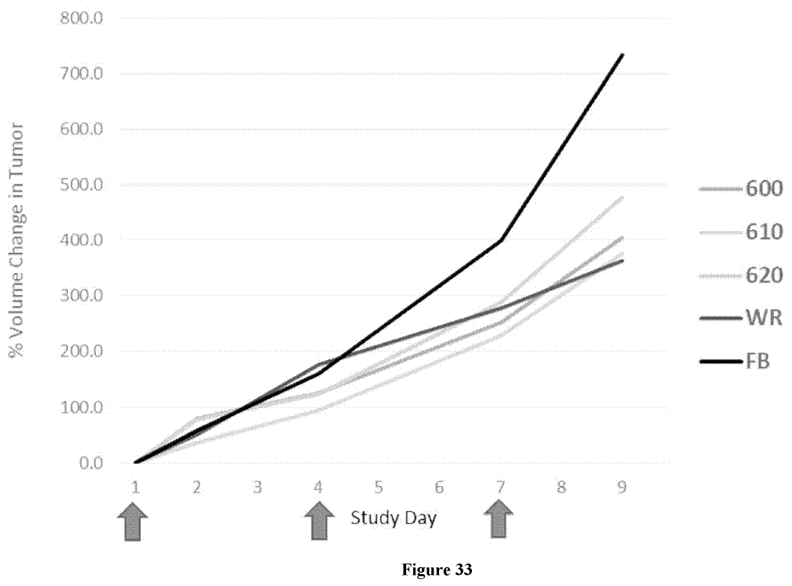

Tumor size changes are recorded and results are summarized in FIG. 33, where % volume change in tumor at day x is calculated by comparing the volume of tumor at day x with the volume of tumor at day 1. FIG. 33 shows that after viral injections at day 1, day 4 and day 7, the increase of the tumor volume size when treated with WR, WR-GS-600, WR-GS-610 and WR-GS-620 is much smaller than that when treated with formulation buffer (FB), suggesting the tumor inhibition effect of the above-mentioned viruses in vivo.

Measurement of Efficacy in Syngeneic Mouse Model

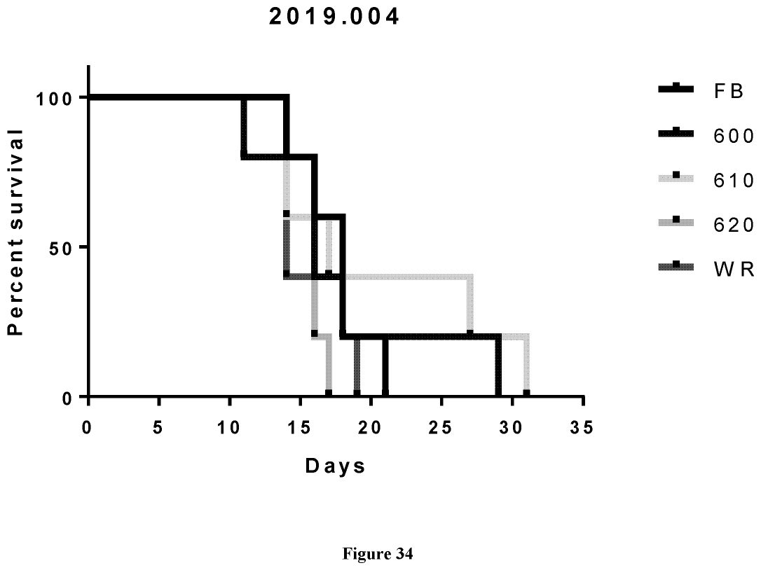

Subcutaneous CT-26LacZ tumor model in Balb/C mice were prepared. Different viruses of 1E7 were injected via tail vein injection at Day 1, 3 and 7, and mice weight and wellness were monitored. Endpoint was set at tumor >1,700 mm³, and study ended at day 31. The mouse survival result is shown in FIG. 34.

Measurement of Tumor Size Change of Humanized HT-29-Luc Subcutaneous Tumor Model after Different Viral Infection

The experiments are described briefly as follows:

Day 1: Order 30 Rag2^{-/-}IL2Rg null mice (Jackson Lab) and implant HT-29 tumor (HT-29 Luc 5E6 cells SQ right flank).

Day 7: IVIS-check tumor growth.

Day 8: Administer via intravenous injection 5.8E6 human PBMC IP.

Day 14: IVIS-assign groups.

Day 15: Treatment 1E7 IT.

Day 18: IVIS and Treatment 1E7 IT.

Day 24: IVIS.

Confirmation of Human Peripheral Blood Mononuclear Cell Engraftment

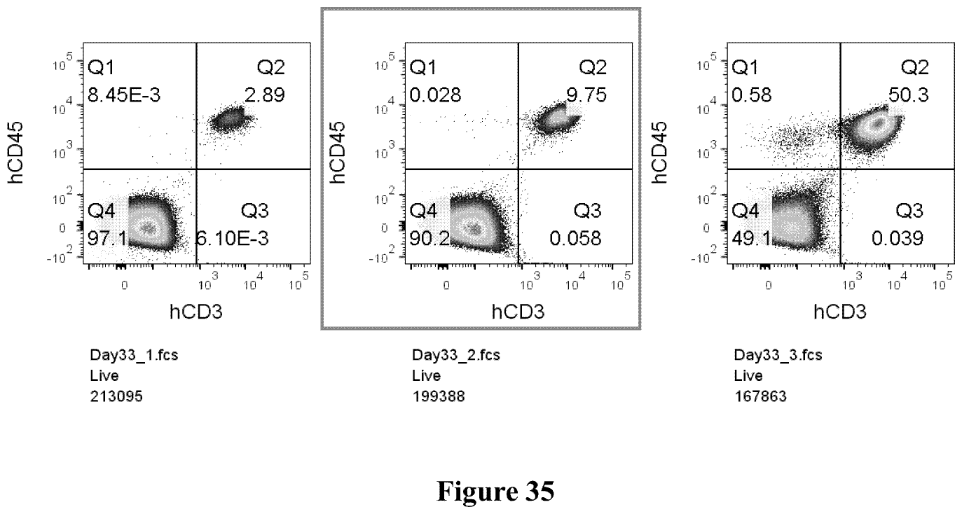

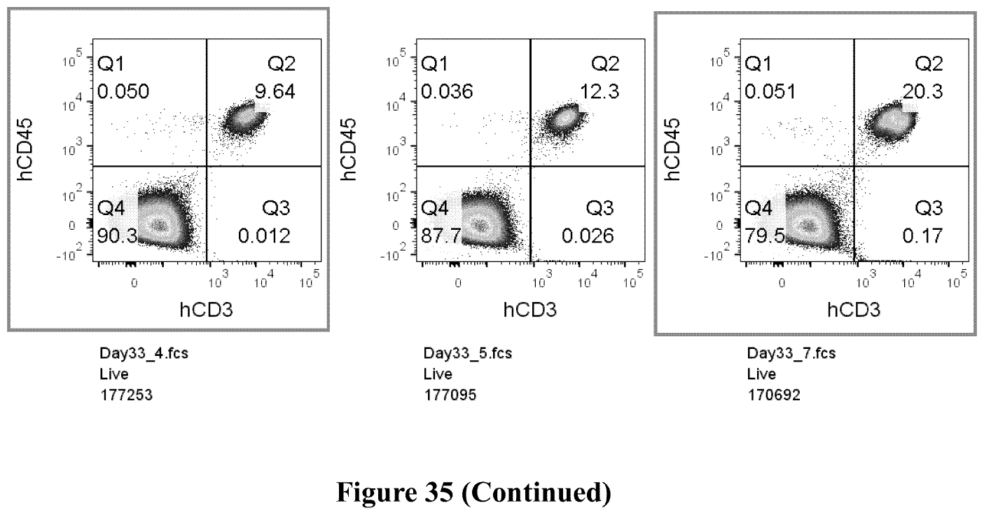

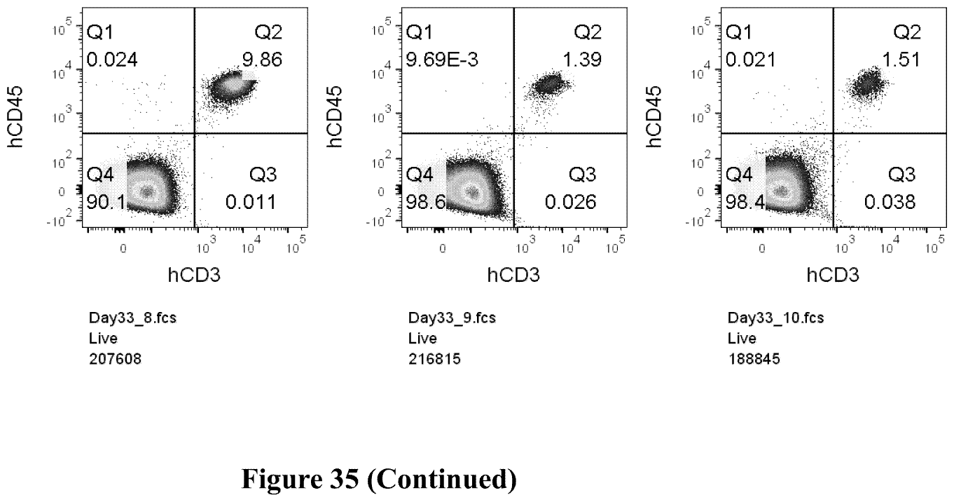

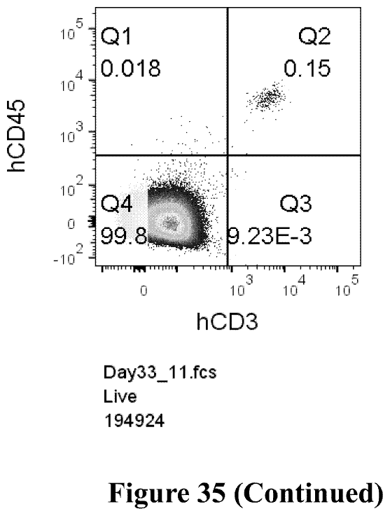

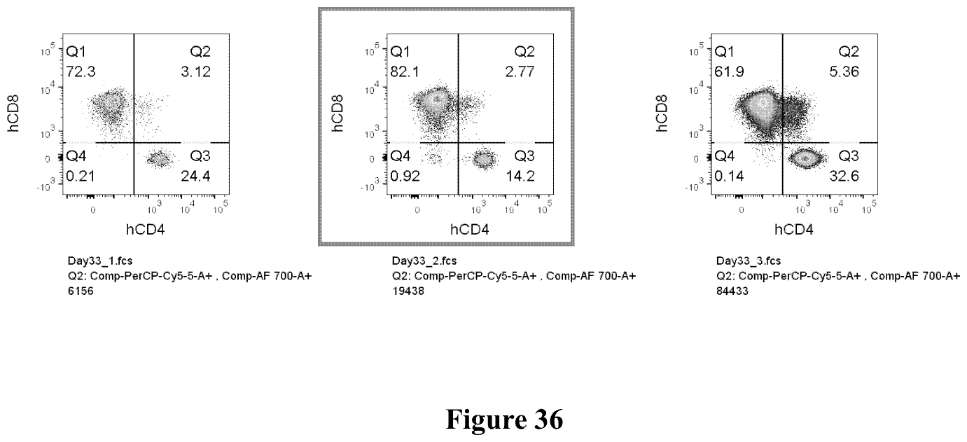

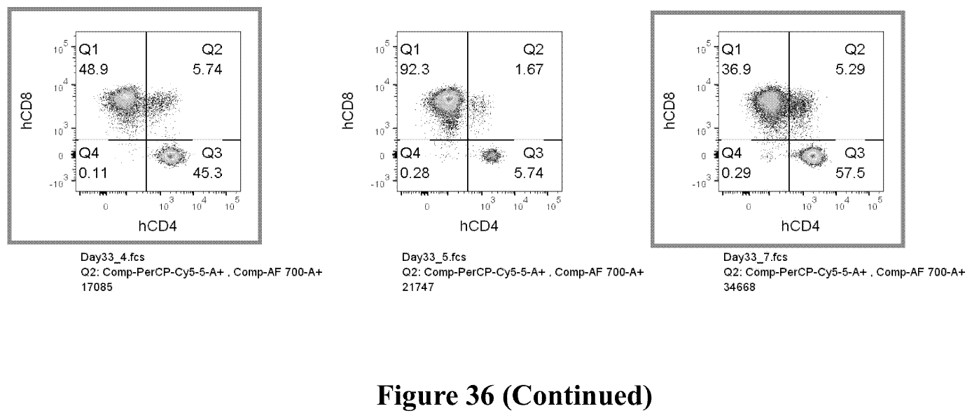

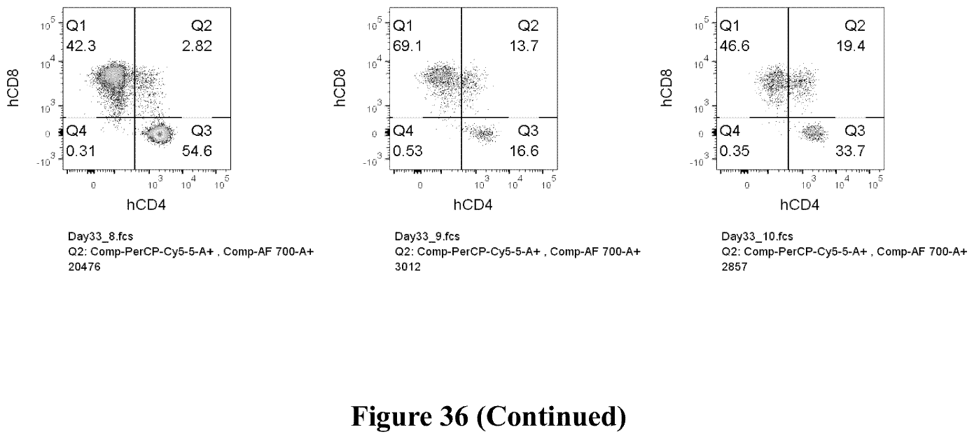

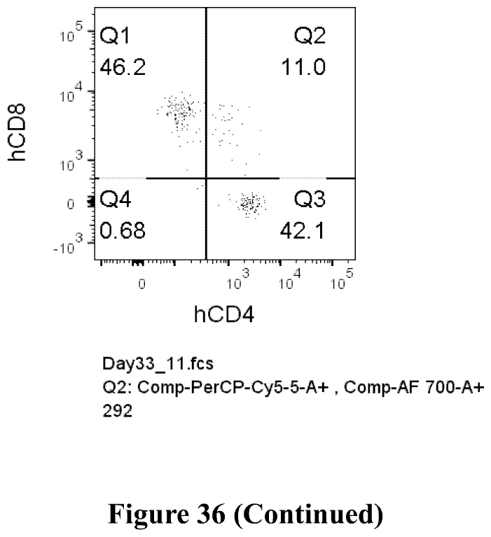

The treated mice were submandibular bled and 100 μL of the blood was obtained and added into sodium heparin. Red blood cells are lysed and stained for hCD45, CD3, CD8 and CD4. The fluorescence results were read on LSR Fortessa and summarized in FIGS. 35 and 36, which confirm that the human peripheral blood mononuclear cells are successfully engrafted into the immunodeficient mice.

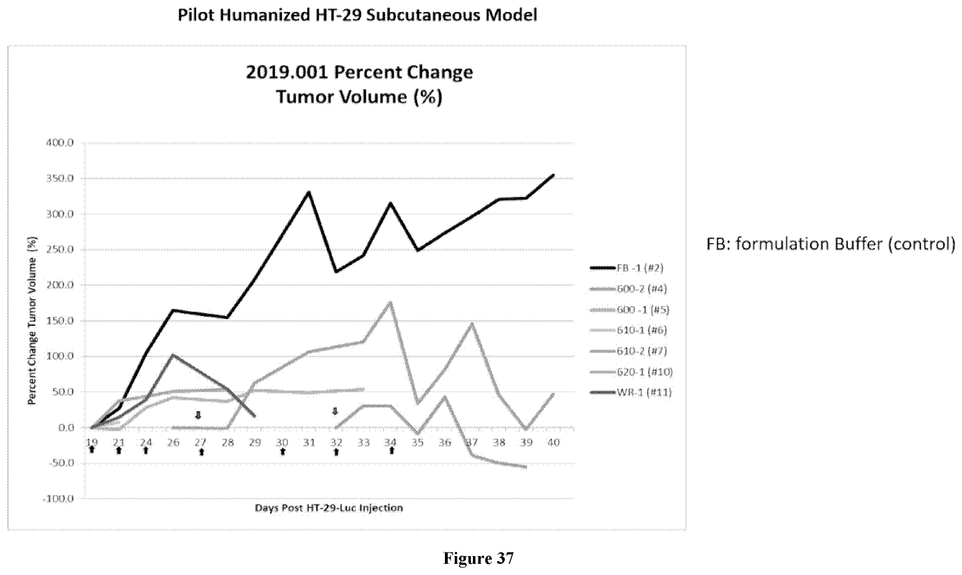

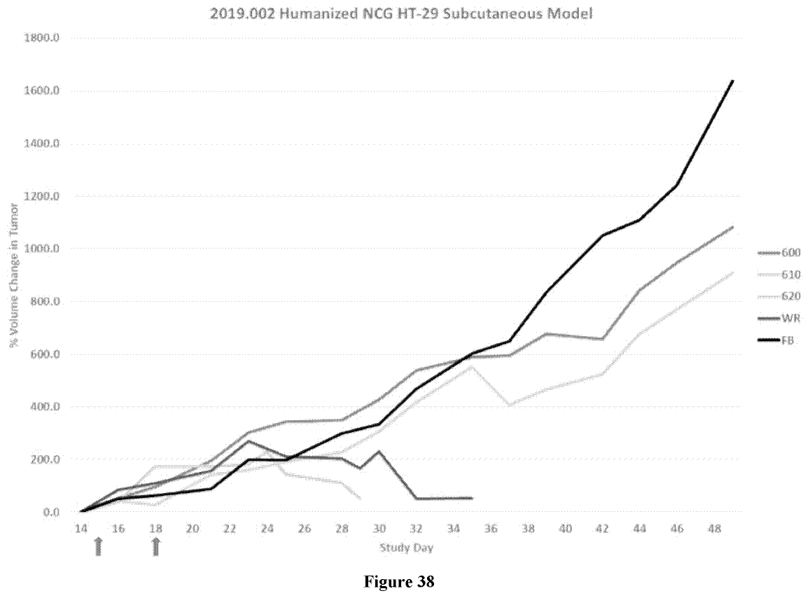

Tumor volume changes after viral infection are summarized in FIGS. 37 and 38. FIG. 37 shows that compared to control group, mice treated with WR-GS-600 exhibits least increase in tumor volume compared to mice treated with WR-GS-610, WR-GS-620, or WR. More interestingly, after infecting mice at day 32 post HT-29-Luc injection with WR-GS-600, the tumor size does not increase significantly and even decreased from day 8 of the WR-GS-600 treatment. FIG. 38 shows that WR and WR-GS-620 have earlier endpoints than WR-GS-600 and WR-GS-610 due to higher toxicity of WR and WR-GS-620 than WR-GS-600 and WR-GS-610. FIG. 38 further shows that WR-GS-600 and WR-GS-610 can control tumor growth when compared to formulation buffer. These data collectively suggest that WR-GS-600 and WR-GS-610 have lower toxicity than WR and WR-GS-620, and both WR-GS-600 and WR-GS-610 can control tumor growth.

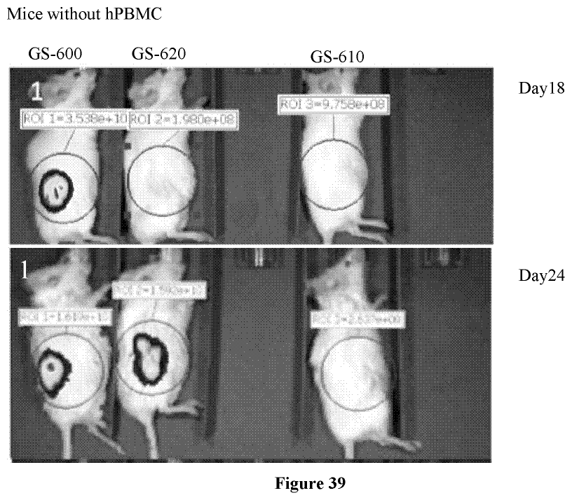

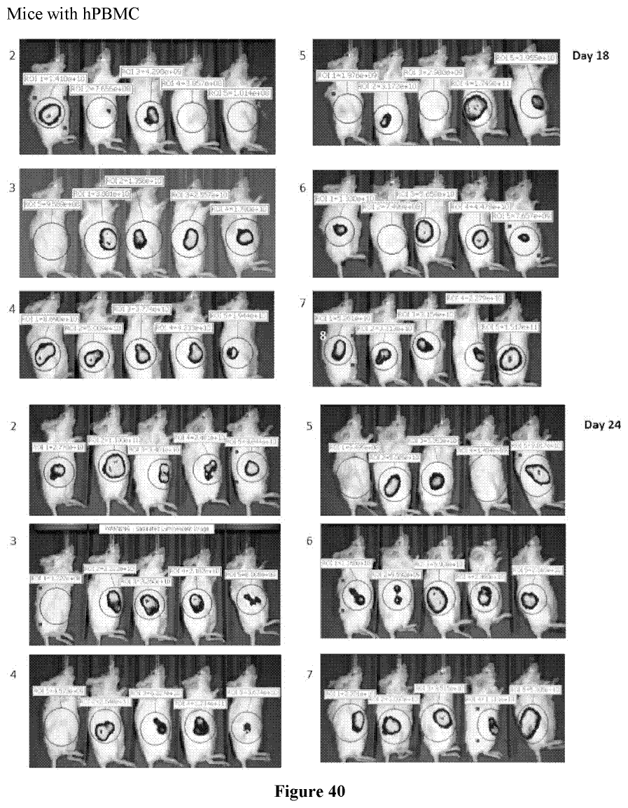

FIGS. 39 and 40 show human tumor HT-29 grow in NCG mice with or without human PBMC through in vivo imaging IVIS measurements (The IVIS spectrum, PerkinElmer).

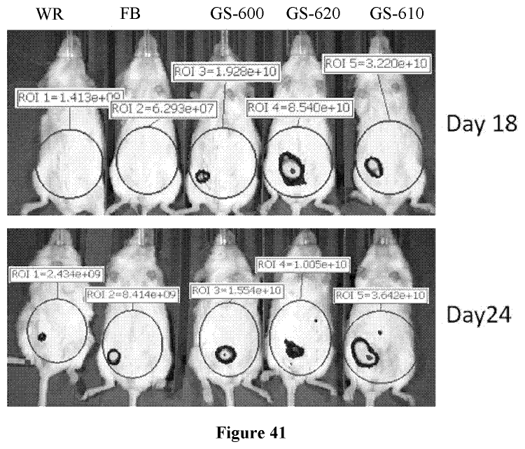

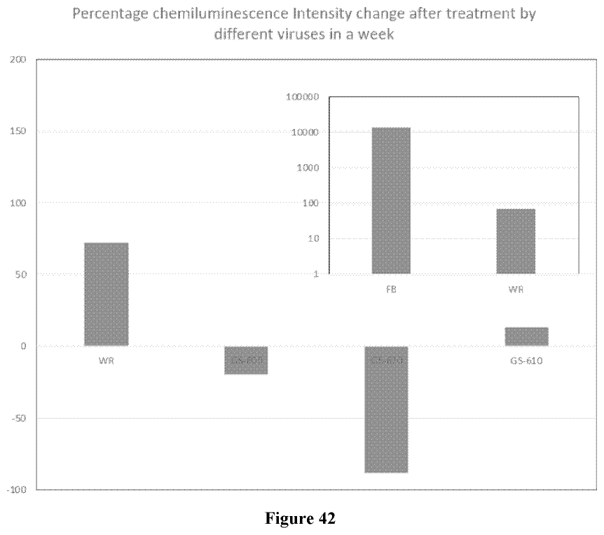

FIGS. 41 and 42 show that for humanized HT-29-Luc intraperitoneal mouse model, where viruses were intraperitoneally injected, WR-GS-600 and WR-GS-620 infection can significantly reduce the chemiluminescence intensity of tumor, suggesting tumor inhibition efficacy of WR-GS-600 and WR-GS-620. Compared to formulation buffer, WR and WR-GS-610 show smaller increase in the chemiluminescence intensity of tumor, suggesting tumor growth control efficacy of WR and WR-GS-610.

The aforementioned in vitro and in vivo results indicate that WR, WR-GS-600, WR-GS-610 and WR-GS-620 can kill cancer cells and control tumor growth. However, WR and WR-GS-620 exhibited higher toxicity, which leads to early termination of drug testing. WR-GS-600 and WR-GS-610 are more effective in tumor growth control, with WR-GS-600 having higher tumor targeting specificity than WR-GS-610. More importantly, in humanized HT-29 intraperitoneal tumor mouse model, intraperitoneal injection of WR-GS-600 decreases tumor size whereas intraperitoneal injection of WR-GS-610 does not stop of increase of tumor size, though the percentage of tumor size increase is much smaller than that when treated with WR. These data suggests that incorporation of both heterologous polynucleotide encoding an immune checkpoint inhibitor and heterologous polynucleotide encoding an immuno activator can reduce toxicity, increase tumor targeting specificity, and improve tumor control efficacy.

With respect to the use of substantially any plural and/or singular terms herein, those having skill in the art can translate from the plural to the singular and/or from the singular to the plural as is appropriate to the context and/or application.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A modified oncolytic virus comprising a virus genome having a first heterologous polynucleotide encoding an immune checkpoint inhibitor and a second heterologous polynucleotide encoding an immuno activator.

2-8. (canceled)

9. The modified oncolytic virus of claim 2, wherein the oncolytic virus is derived from the Western Reserve strain.

10. The modified oncolytic virus of claim 1, wherein the immune checkpoint inhibitor is a first antibody capable of specifically binding to an immune checkpoint protein or the antigen binding fragment thereof, wherein the immune checkpoint protein is selected from a group consisting of PD-1, PD-L1/2, CTLA-4, B7-H3/4, LAG3, TIM-3, VISTA and CD160.

11-12. (canceled)

13. The modified oncolytic virus of claim 10, wherein the first antibody or the antigen binding fragment thereof comprises a first heavy chain comprising SEQ ID NOs: 2, 3, and 4, and the first antibody or the antigen binding fragment thereof further comprises a first light chain comprising SEQ ID NOs: 9, 10, and 11.

14-15. (canceled)

16. The modified oncolytic virus of claim 13, wherein the first heavy chain comprises an amino acid sequence of SEQ ID NO: 6 or a homologous sequence thereof having at least 80% sequence identity, and the first light chain comprises an amino acid sequence of SEQ ID NO: 13 or a homologous sequence thereof having at least 80% sequence identity.

17. The modified oncolytic virus of claim 11, wherein the first heterologous polynucleotide comprises a nucleic acid sequence of SEQ ID NO: 8 or a homologous sequence thereof having at least 80% sequence identity, and the first heterologous polynucleotide further comprises a nucleic acid sequence of SEQ ID NO: 15 or a homologous sequence thereof having at least 80% sequence identity.

18-22. (canceled)

23. The modified oncolytic virus of claim 1, wherein the immuno activator is a second antibody binding to a co-stimulatory molecule or the antigen binding fragment thereof, wherein the co-stimulatory molecule is selected from a group consisting of CD137 (4-1BB), CD27, CD70, CD86, CD80, CD28, CD40, CD122, CD27/70, TNFRS25, OX40, GITR, Neutrophilin and ICOS.

24-25. (canceled)

26. The modified oncolytic virus of claim 23, wherein the second antibody or the antigen binding fragment thereof comprises a second heavy chain comprising SEQ ID NOs: 17, 18, and 19 and the second antibody or the antigen binding fragment thereof further comprises a second light chain comprising SEQ ID NOs: 24, 25, and 26.

27-28. (canceled)

29. The modified oncolytic virus of claim 26, wherein the second heavy chain comprises an amino acid sequence of SEQ ID NO: 21 or a homologous sequence thereof having at least 80% sequence identity and the second heterologous polynucleotide further comprises a nucleic acid sequence of SEQ ID NO: 29 or a homologous sequence thereof having at least 80% sequence identity.

30. The modified oncolytic virus of claim 23, wherein the second heterologous polynucleotide comprises a nucleic acid sequence of SEQ ID NO: 23 or a homologous sequence thereof having at least 80% sequence identity and the second light chain comprises an amino acid sequence of SEQ ID NO: 28 or a homologous sequence thereof having at least 80% sequence identity.

31-41. (canceled)

42. The modified oncolytic virus of claim 1, wherein the immune checkpoint inhibitor is an antibody specifically binding to PD-1 or the antigen binding fragment thereof, and the immuno activator is an antibody specifically binding to CD137 or the antigen binding fragment thereof.

43. The modified oncolytic virus of claim 4, wherein the first heterologous polynucleotide and the second heterologous polynucleotide is inserted in the place of the deletion.

44. The modified oncolytic virus of claim 43, wherein the first heterologous polynucleotide is immediately upstream or immediately downstream of the second heterologous polynucleotide.

45. (canceled)

46. The modified oncolytic virus of claim 45, wherein the first heterologous polynucleotide further comprises a first promoter capable of driving expression of the first heavy chain, and a second promoter capable of driving expression of the first light chain, wherein the first and the second promoters are in a head-to-head orientation.

47. (canceled)

48. The modified oncolytic virus of claim 47, wherein the second heterologous polynucleotide further comprises a third promoter capable of driving expression of the second heavy chain, and a fourth promoter capable of driving expression of the second light chain, wherein the third and the fourth promoters are in a head-to-head orientation.

49-55. (canceled)

56. The modified oncolytic virus of claim 1, wherein the modified oncolytic virus comprises the following elements in frame in an orientation from 5′ to 3′ of the sense strand: a polynucleotide encoding the light chain of an antibody binding to CD137-a first early and late promoter-a second early and late promoter-a polynucleotide encoding the heavy chain of an antibody binding to CD137-a polynucleotide encoding the heavy chain of an antibody binding to PD-1-a first late promoter-a second late promoter-a polynucleotide encoding the light chain of an antibody binding to PD-1.

57. The modified oncolytic virus of claim 1, wherein the immune checkpoint inhibitor expressed from the first heterologous polynucleotide and the immuno activator expressed from the second heterologous polynucleotide are expressed as separate proteins.

58. A pharmaceutical composition, comprising the modified oncolytic virus of claim 1 and a pharmaceutically acceptable carrier.

59. A method of treating a tumor, comprising administering to a subject an effective amount of the modified oncolytic virus of claim 1.

60-61. (canceled)

62. The method of claim 59, wherein the tumor is melanoma, non-small cell lung cancer, renal cell carcinoma, Hodgkin lymphoma, squamous cell carcinoma of the head and neck, bladder cancer, colorectal cancer, or hepatocellular carcinoma.

63-64. (canceled)


SEQUENCE LISTING (46 sequences)

SEQ ID NO: 1 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 5 aa, Artificial Sequence
  Synthetic
NSGMH

SEQ ID NO: 3 - protein, 17 aa, Artificial Sequence
  Synthetic
VIWYDGSKRYYADSVKG

SEQ ID NO: 4 - protein, 4 aa, Artificial Sequence
  Synthetic
NDDY

SEQ ID NO: 5 - protein, 113 aa, Artificial Sequence [sequence omitted]

SEQ ID NO: 6 - protein, 440 aa, Artificial Sequence
  Synthetic
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 339 bp, Artificial Sequence
  Synthetic
cgaacttacggtcaccaatgtgccctgtccccagtaatcgtcgtttgtggcgcagtagtatacggctgtgtcctcagcccgcagtgaattcatctgtaggaacaatgtatttttagagttgtccctactaatggtgaaccgtcccttcacagagtcggcgtaataacgcttggagccgtcataccagataactgccacccactccaagcccttgcctggcgcttgccgtacccagtgcatcccgctgttagaaaaggtgatgccggaagccttacagtccaatcttagtgatctgcctggctggacaactccgccgcctgactcaaccagctgtacttg

SEQ ID NO: 8 - DNA, 890 bp, Artificial Sequence
  Synthetic
ctgacgacccgataggttgaattgaattgttcctcgcggggtttcgtctttgcgttatgcacttcaacgccgtctacgtaccagttgaattgcacttcgggatcctcttgagagacatcaaccacgacacaagtcacttccggggtccggctaatcattagggtgtccttaggttttggtgggaacagaaacacggacggtccacccaagaactcgggggcaggacacggagggcaggggggtccatacttggattcgacccgcttatctactttagtattagagggcttgtggtcaacgttacaggtataagttttcgtgcctagactagatgaaggaacagtgaccacggaagatagtgaatagaggcccgagctctgcagcacggcagggaatgtgtgcactccagaggtcagggcgccgctgttccaggacacagtcaccggttctggaaagtagtccttcacgagacatcctagcgcagctgtgctttcgcttgtgctcctactgcagggggcgagcgggaacactgagggtcccttggtgcttgccgaacttacggtcaccaatgtgccctgtccccagtaatcgtcgtttgtggcgcagtagtatacggctgtgtcctcagcccgcagtgaattcatctgtaggaacaatgtatttttagagttgtccctactaatggtgaaccgtcccttcacagagtcggcgtaataacgcttggagccgtcataccagataactgccacccactccaagcccttgcctggcgcttgccgtacccagtgcatcccgctgttagaaaaggtgatgccggaagccttacagtccaatcttagtgatctgcctggctggacaactccgccgcctgactcaaccagctgtacttg

SEQ ID NO: 9 - protein, 11 aa, Artificial Sequence
  Synthetic
RASQSVSSYLA

SEQ ID NO: 10 - protein, 7 aa, Artificial Sequence
  Synthetic
DASNRAT

SEQ ID NO: 11 - protein, 9 aa, Artificial Sequence
  Synthetic
QQSSNWPRT

SEQ ID NO: 12 - protein, 107 aa, Artificial Sequence [sequence omitted]

SEQ ID NO: 13 - protein, 214 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 320 bp, Artificial Sequence
  Synthetic
gaaatcgtactcacgcagtcccctgctactctgagtctctcaccaggagaacgcgctaccctttcttgccgtgcgtcacagtcagtatcgtcctatctggcttggtatcagcaaaaaccaggtcaggccccccgattattgatttatgatgcatctaaccgggctacagggattcctgccagatttagcggtagcgggagtggaactgacttcactctaacattagctcccttgagccagaggatttcgccgtctactactgtcagcagtcttccaactggcctcgtactttcggacagggaacaaaggtggaaatcaaa

SEQ ID NO: 15 - DNA, 642 bp, Artificial Sequence
  Synthetic
gaaatcgtactcacgcagtcccctgctactctgagtctctcaccaggagaacgcgctaccctttcttgccgtgcgtcacagtcagtatcgtcctatctggcttggtatcagcaaaaaccaggtcaggccccccgattattgatttatgatgcatctaaccgggctacagggattcctgccagatttagcggtagcgggagtggaactgacttcactctaaccattagctcccttgagccagaggatttcgccgtctactactgtcagcagtcttccaactggcctcgtactttcggacagggaacaaaggtggaaatcaaacgtaccgtggctgcacccagcgtgttcatttttccaccaagcgacgagcagctcaagagcggaaccgcatccgtagtatgtctcctcaataacttctacccacgagaagccaaagtgcagtggaaggtggataatgccttgcaatccggaaacagccaagaaagcgtgaccgaacaggattcaaaagacagcacctattctctgtccagcacattgacactgagtaaagctgattatgagaagcacaaggtctacgcgtgtgaggttacacatcaaggattgtcttcaccagtcaccaagagtttcaatagaggagagtgc

SEQ ID NO: 16 - protein, 238 aa, Homo sapiens
FERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 17 - protein, 5 aa, Artificial Sequence
  Synthetic
GYYWS

SEQ ID NO: 18 - protein, 16 aa, Artificial Sequence
  Synthetic
EINHGGYVTYNPSLES

SEQ ID NO: 19 - protein, 13 aa, Artificial Sequence
  Synthetic
DYGPGNYDWYFDL

SEQ ID NO: 20 - protein, 121 aa, Artificial Sequence [sequence omitted]

SEQ ID NO: 21 - protein, 448 aa, Artificial Sequence
  Synthetic
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - DNA, 363 bp, Artificial Sequence
  Synthetic
caggttcagctacagcagtggggagcaggtctgctgaaaccaagcgaaactttgtccttgacgtgtgcagtctatggagggtcctttagcggttattattggtcctggattagacagtcccctgagaaggggctcgagtggataggcgaaatcaatcatggggggtatgtgacttacaatccctccctcgagtcccgggtaactatcagcgtggacacttcgaagaatcaattttcactaaagcttagttctgtcactgctgctgatacagctgtctactattgtgcgcgtgattacggaccaggtaattacgattggtatttcgacttgtgggggaggggtaccttggtcacagtatcatcc

SEQ ID NO: 23 - DNA, 1344 bp, Artificial Sequence
  Synthetic
caggttcagctacagcagtggggagcaggtctgctgaaaccaagcgaaactttgtccttgacgtgtgcagtctatggagggtcctttagcggttattattggtcctggattagacagtcccctgagaaggggctcgagtggataggcgaaatcaatcatggggggtatgtgacttacaatccctccctcgagtcccgggtaactatcagcgtggacacttcgaagaatcaattttcactaaagcttagttctgtcactgctgctgatacagctgtctactattgtgcgcgtgattacggaccaggtaattacgattggtatttcgacttgtgggggaggggtaccttggtcacagtatcatccgcaagtacgaagggcccttccgtgtttccactcgctccctgcagtcgaagcacctcagaatcaaccgccgctctggggtgtctcgtgaaggactacttcccggaacctgtgaccgtcagctggaactccggggccctgacgagcggagtgcacaccttccccgccgtgctccagagtagtggactttactccttatcttccgtcgtcacagtgcctagttcatctctggggaccaagacatacacttgcaacgtggaccataaaccttcaaacacgaaagtcgataaacgcgtcgagtctaaatacggtcctccatgtccgccttgccctgcccccgagtttctaggaggaccatcagtctttcttttcccaccaaaaccgaaggacacgctcatgatttcacggacccccgaagtgacctgcgtggtggtggacgtatcccaggaggatccagaggtgcagtttaattggtatgtggacggggtagaagttcataacgctaaaacgaagcctcgcgaggaacaattcaatagtacctatagagtggtgtcagtgctcactgtactgcaccaggattggctcaacggcaaggagtacaaatgtaaggtgtccaataaggggctgcccagttctattgagaagacaatcagcaaggcgaagggccagccaagggagccacaggtctatacactaccaccaagccaggaagagatgacaaagaaccaggtgtcactgacttgtctggtcaagggcttttatccatctgatattgccgtggagtgggagtccaacggacagccagagaacaactacaagaccaccccccccgtcctggactctgacggctcattctttctgtatagcagactgaccgtggataagtctcggtggcaggaagggaacgtcttctcgtgcagcgtcatgcacgaggccctgcacaaccactacacgcagaagtctctctcgctttccctagggaag

SEQ ID NO: 24 - protein, 11 aa, Artificial Sequence
  Synthetic
RASQSVSSYLA

SEQ ID NO: 25 - protein, 7 aa, Artificial Sequence
  Synthetic
DASNRAT

SEQ ID NO: 26 - protein, 11 aa, Artificial Sequence
  Synthetic
QQRSNWPPALT

SEQ ID NO: 27 - protein, 109 aa, Artificial Sequence [sequence omitted]

SEQ ID NO: 28 - protein, 327 aa, Artificial Sequence
  Synthetic
TTTAATTTCCACCTTGGTACCACCACCGAACGTCAGAGCGGGAGGCCAATTAGACCTCTGTTGACAATAGTAAACTGCGAAATCCTCGGGTTCCAACGAACTAATGGTCAGTGTGAAGTCGGTGCCGCTGCCGGATCCGGAAAACCTGGCTGGGATGCCGGTGGCCCGATTGGACGCGTCATAGATCAGTAGTCTGGGAGCCTGACCCGGTTTCTGCTGATACCAAGCCAGATATGATGAAACGCTCTGGGATGCCCTGCAGGATAAGGTTGCACGCTCCCCTGGAGACAGACTCAGGGTTGCTGGGGACTGCGTCAGTACTATCTC

SEQ ID NO: 29 - protein, 216 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 648 bp, Artificial Sequence
  Synthetic
gcactcgcctcgattgaagctcttagtaacggggctggataagccctggtgtgtcacttcgcaggcatacaccttatgtttctcgtagtcggccttactcagggtcagggttgaagaaagtgaatacgtagaatctttggagtcttgctcggtcacgctctcttgactattcccgctctgcagggcattgtccaccttccattggacttttgcctccctggggtaaaagttgttgaggagacagacgacgctcgccgtgccggacttgagctgttcgtcagatggagggaagatgaaaacgctgggagccgcgacggttcttttaatttccaccttggtaccaccaccgaacgtcagagcgggaggccaattagacctctgttgacaatagtaaactgcgaaatcctcgggttccaacgaactaatggtcagtgtgaagtcggtgccgctgccggatccggaaaacctggctgggatgccggtggcccgattggacgcgtcatagatcagtagtctgggagcctgacccggtttctgctgataccaagccagatatgatgaaacgctctgggatgccctgcaggataaggttgcacgctcccctggagacagactcagggttgctggggactgcgtcagtactatctc

SEQ ID NO: 31 - DNA, 30 bp, Artificial Sequence
  Synthetic
atggatcacaaccagtatctcttaacgatg

SEQ ID NO: 32 - DNA, 30 bp, Artificial Sequence
  Synthetic
gaaatatagattgttgtagaaatagtacct

SEQ ID NO: 33 - DNA, 24 bp, Artificial Sequence
  Synthetic
atatcgcattttctaacgtgatgg

SEQ ID NO: 34 - DNA, 24 bp, Artificial Sequence
  Synthetic
ggtttatctaacgacacaacatcc

SEQ ID NO: 35 - DNA, 24 bp, Artificial Sequence
  Synthetic
gatgcgattcaaaaaagaatcctc

SEQ ID NO: 36 - DNA, 24 bp, Artificial Sequence
  Synthetic
ggataaggttgcacgctcccctgg

SEQ ID NO: 37 - DNA, 24 bp, Artificial Sequence
  Synthetic
ctttactccttatcttccgtcgtc

SEQ ID NO: 38 - DNA, 24 bp, Artificial Sequence
  Synthetic
gcaacgcttcgtgcatcacggagc

SEQ ID NO: 39 - DNA, 24 bp, Artificial Sequence
  Synthetic
gtagtccttcacgagacatcctag

SEQ ID NO: 40 - DNA, 24 bp, Artificial Sequence
  Synthetic
gccgtctactactgtcagcagtct

SEQ ID NO: 41 - DNA, 25 bp, Artificial Sequence
  Synthetic
tgtgtaccgggagcagatcctatat

SEQ ID NO: 42 - DNA, 24 bp, Artificial Sequence
  Synthetic
cggcgcagtgagtaatcaaggtca

SEQ ID NO: 43 - DNA, 24 bp, Artificial Sequence
  Synthetic
attagccggaccccggaagtgact

SEQ ID NO: 44 - DNA, 24 bp, Artificial Sequence
  Synthetic
ggcttggtggtagtgtatagacct

SEQ ID NO: 45 - DNA, 24 bp, Artificial Sequence
  Synthetic
accccccatgattgatttcgccta

SEQ ID NO: 46 - DNA, 24 bp, Artificial Sequence
  Synthetic
ctccaaagattctacgtattcact